(19) United States
(12) Patent Application Publication
(10) Pub. No.: US 2011/0319944 A1
(43) Pub. Date: Dec. 29, 2011

(54) Submuscular Facial Fixation (Myo-Osseous Fixation) Using Microincision Microscrew Device, Injectable Glues and Adhesives, and Method and Device for Therapy of Migraine and Related Headaches

(76) Inventor: Gary E. Borodic, Quincy, MA (US)

(21) Appl. No.: 13/109,925

(22) Filed: May 17, 2011

Related U.S. Application Data
(63) Continuation-in-part of application No. PCT/US2009/006219, filed on Nov. 20, 2009.
(60) Provisional application No. 61/117,069, filed on Nov. 21, 2008.
(60) Provisional application No. 61/344,067, filed on May 17, 2010.

(51) Int. Cl. A61B 17/86; A61B 17/00

(52) U.S. Cl. 606/301; 606/151

(57) ABSTRACT

Procedures for altering the surface of the human face or other body parts by tethering muscle origins, extending muscular attachments and origins, and tethering muscles by causing adhesions of facial muscles which are not naturally present using specific methods materials and devices, and procedures for treating headaches by implanting a device through a micro incision in skin is are described. A microincision microscrew device and methods of using the device in surgical procedures is described. The use of a puncture based injection of bioadhesive into a described functional fibrofatty plane on the undersurface and throughout the muscle proper without use of incisional surgery is also described. Osseous bolting and osseous screws fixating soft tissue elevation via deep muscle tethering to facial bone is shown causing suspension of upper, mid, and lower facial soft tissue structures in a method that is adaptable to other body regions. Additionally, the combined use of screws with bioadhesives and single use of injectable bioadhesives are described.

### RELATED APPLICATIONS

This application is a continuation in part of copending international application number PCT/US2009/06219, filed Nov. 20, 2009, which claims priority to U.S. Provisional Application No. 61/117,069 filed Nov. 21, 2008, the disclosures of which are herein incorporated by reference in their entireties. This application also claims priority to copending U.S. Provisional Application No. 61/344,067, filed May 17, 2010, the disclosure of which is herein incorporated by reference in its entirety.

### FIELD OF THE INVENTION

The present invention is directed to a method and supporting devices to accomplish soft tissue fixation in the face or other body regions for the purpose of treating movement disorders as well as accomplishing alteration of facial contour and position relative to supporting facial bone or elevating or contouring other body regioins, as well as for treating migraines and related headaches. A microincision microscrew device and methods of using the device in surgical procedures is described. Described herein are methods of osseous bolting and osseous screws for soft tissue elevation via deep facial plate compression fixation; suspension of upper, mid, and lower facial soft tissue structures; and other body regions. Described herein also are injectable materials that require puncture and no incision to accomplish suspension of upper, mid, and lower facial soft tissue structures; and other body regions. Some embodiments of the present invention involve either a microincision or puncture as opposed to conventional larger tissue plan dissections. This results in rapid healing, reduced recovery period, and ease of use for the treated subject and the surgeon or physician. Some embodiments of the invention use the concept that the a planar joint lies on the undersurface of facial and other muscles consisting of fibro fatty tissue which provides a low resistance plane for movement. This plane is targeted for alteration by screw or adhesive material to elevate or proportionally alter, for example, cheek, jowl, and brow over facial bones, therefore creating a more vital and youthful appearance. Also described herein are methods and devices for the therapy of migraine and related headaches. In some embodiments of the invention a device is surgically implanted through a micro incision through skin that causes fixation of the dermis and muscle elements to bone, which desensitizes the trigemenial nerve.

In some embodiments of the invention, methods to treat chronic recurrent migraines and related headaches, such as severe myofascial pain, cluster headaches, cervciogenic headaches, and headaches associated with fibromyalgia, are described. In other embodiments, methods to treat chronic severe tension and related headaches, recurrent cluster headaches, migraine and associated headaches, or photophobia are described. In other embodiments, methods to treat headaches in classes 1 to 13 of the 1988 Headache and Facial Pain Classification System by the International Headache Society are described.

### BACKGROUND

Facelift, brow lift, and mid facelift are all cosmetic procedures practiced and advocated by plastic surgeons for aesthetic improvement. These procedures involve various methods of subcutaneous tightening of the facial plane to achieve elevation of critical structures of the face such as the brow, jowl region, and cheek areas. Such elevations produce a more youthful and aesthetically pleasing appearance.

Previous methods of achieving such suspensions and soft tissue elevations have included the use of sling procedures with various sutures; tightening of the superficial muscular aponeurotic system (SMAS) (see FIG. 1); resection of regions of the brow; skin resections; as well as various forms of tightening procedures using thermal cautery, or transcutaneous cautery such as Thermage or ultrasonic collagen contraction. Brow lifts are often done by use of an endoscope with internal peri-orbital orbital muscle cutting and large dissection planes, often requiring general anesthesia, extended operating room time, and an extended post operative recovery time.

Surgeons in the past have used suspension or hook-type implants screwed to the skull that use a hook mechanism or projection device to suspend soft tissue from the point of osseous tissue fixation (see Coapt Technologies, Inc). These prior methods are inadequate because they have been noted to be notoriously irreproducible, and moreover, they often do not produce lasting results and/or cause tissue inflammatory reaction from the implanted materials. Because an incision is needed, these devices are also associated with varying degrees of scarring. More invasive procedures require more extensive tissue dissections, which results in more inflammation, scarring, longer healing time and increased pain and suffering.

Use of glues have been advocated for conventional facelift surgery and other forms of facial plastic surgery. However, using glue to actually replace incision surgery has never been contemplated before the present invention. In one embodiment of the present invention, glue or bioadhesives are used as an injectable delivered into a critical anatomic tissue plane, i.e., between bone and the undersurface of muscle, followed by external manipulation. In this implementation, the need for an incision is eliminated or radically reduced.

Migraine and tension headaches are some of the most common afflictions experienced by the human race. Migraine has been thought to occur in 20% of the female population. Migraine is the most common cause for employment absenteeism, only behind the common cold and flu. The pain associated with migraine can be severe and devastating, causing complete incapacitation. Other headache syndromes related to migraine can include cluster headache, atypical facial neuralgia, temporomandibular joint disease, myofascial pain headache, fibromyalgia, sinus migraine, as well as many other forms of headaches not associated with structural or pathologic lesions listed in the International Headache Society (IHS) Classification of human headache disorders published and referenced herein.

The migraine is characterized most specifically as a recurring, intermittent, episodic headache lasting usually between 4 and 72 hours, most often associated with photophobia (light sensitivity), phonophobia (sound sensitivity), pain worsening with movement, severe throbbing elements often unilateral, and most often associated with some element of nausea and sometimes repeated vomiting. The severity of the pain often causes cessation of routine daily activities and occasionally leads to emergency room visits for the use of parenteral narcotics or other pain relieving drugs. The temporal headache is often recurring. The intensity of the problem often depends on the number of headaches experienced by afflicted individuals. This can range anywhere from several a week to one or two a year. When migraine occurs frequently upregulation of peripheral nociceptors and central pain centers in the brain can lead to the occurrence of chronic daily headache associated with the episodic migraine, leading to almost a permanent form of headache disorder. Most often there is a prodromal period present in which the patient feels an impending headache is about to initiate prior to actually experiencing any degree of pain. This prodromal period is important to the application of the invention described herein, in that it allows the patient a signal to the onset of the headache, and the opportunity to apply therapy to abort the full evolution into an explosive headache. This period can occur over a period of minutes to hours. About 15% of afflicted patients actually have what is known as a true aura, usually seeing flashing lights which may precede the pain. The prodrome and the aura allow again for the opportunity for the institution of abortive therapy to prevent the initiation of the severe, painful phase of the headache.

Treatment can involve addressing the severe pain with analgesics. When the pain is extreme the use of narcotics, such as codeine, OxyContin, Oxycodone and Demerol, or nonsteroidal anti-inflammatory analgesics (NSAIDs), such as Toradol, can be used. Other medications used include barbiturates (eg Phenobarbital, Furocet). These medications are sometimes associated with the use of ice packs. Narcotic medications can be addicting if used frequently. They often promote nausea, which aggravates the preexisting tendency for nausea which occurs with migraine.

There are abortive drugs in the classifications of medications used to treat migraine, such as the use of Ergot alkaloids which can be given orally, nasally, intravenously, or rectally. Abortive therapy has most recently added the triptans, which are given by mouth in the form of a pill, nasal spray, or self administered injection, into the arm or thigh. The use of Imitrex (sumatriptan) was the leading drug in this category of families. These drugs are serotonin acting, working on activating 5HT3 receptors. The 5HT3 receptor has been associated with the mechanism of action of the triptan family. Agonist agents have been established in mitigating migraine headache. Side effects of triptan drugs have included cardiovascular events including arrhythmias, coronary vasospasm, possible myocardial infarction, and even death.

Another category of medications used has been prophylactic therapies, which are usually given on a daily basis to decrease the frequency of the headache disorder. These are not drugs generally given during the episode, but are given on a regular basis. Such medications can include, but are not limited to the use of aspirin, antiseizure drugs such as Neurontin, methylsergide, beta-blocking agents, anti calcium channel blockers such as verapamil, and tricyclic antidepressant drugs such as amitriptyline and nortriptyline. These drugs have been also associated with side effects. Most recently the use of botulinum toxin has been used as a prophylactic agent because of its long duration of action, 3 to 4 months. This drug is injected through the skin and the forehead and temporal and posterior cervical region which also has been shown in a number of clinical studies to be protective against frequency of headaches. This drug is under current FDA phase 3 trials, sponsored by Allergan pharmaceuticals. It is designed to achieve approval as a prophylactic agent.

Over the counter medications have classically been used to treat migraine headaches and other forms of headaches. These are most available to patients and include drugs such as Indocin, aspirin, Motrin, and other drugs known as nonsteroidal, anti-inflammatory agents. These agents usually work on the prostaglandin synthesis system, which helps relieve local release of prostaglandin and related mediators which can be involved in sensitization and inciting pain. These pharmaceuticals are not very effective in severe migraine or chronic daily muscle tension headache. Some of these agents when used chronically can be associated with rebound headache if stopped; therefore a functional addiction can occur.

The use of triptan related abortive agents has been associated with cardiovascular complications (arrhythmias, vasospasm, chest pain, possible myocardial infarction). The triptan family can be used as an oral pill, nasal inhaler, or as a self administered injection. Another abortive agent class includes the ergot alkaloids. These agents have also been associated with vascular complications inclusive of cardiovascular complications, coronary vasospasm, peripheral vasospasm and gangrene.

Another class of anti-migraine therapy include the prophylactic drugs. Beta blockers are among the best known which include propranolol and timolol. Beta-blockers have been associated with inducing asthma and blocking the natural release of insulin during hypoglycemia. Antiseizure agents, such as Neurontin, Depakote, and Topamax have also been associated with side effects. Verapamil can cause constipation, fatigue, muscle pain. Topamax can be associated with weight loss and changes in appetite, and Depakote, approved for migraine headache prophylaxis, has been associated with birth defects, a serious complication given that migraine headaches are most commonly encouraged at childbearing age of a woman's life. The tricyclic antidepressants are associated with dry mouth, constipation, visual blurring, as well as other autonomic side effects. Fioricet and Fiorinal are often prescribed medications which contain phenobarbital. Barbiturates are an addicting form of drugs, and the chronic use of these medications can lead to, as with the narcotics, an addiction problem to these agents. The corticosteroids have been occasionally used as prophylaxis; these also have many side effects inclusive of fluid retention, osteoporosis, psychic changes, GI disturbances, skin changes, and reconfiguration of body fat. In essence all oral agents used to treat human headache disorders on a chronic basis have had some degree of side effects and again can often be ineffective.

### SUMMARY OF THE INVENTION

Embodiments of the invention described herein are directed to novel methods of fixating soft tissue to bone structures wherein the methods do not involve a hook mechanism or projection device to suspend the soft tissue. Instead, embodiments of the instant invention are directed to a bolt or a screw-type device that is placed directly under a deep facial plane, such as the gala of the scalp, or the SMAS or suborbicularis orbital fascia or other portion of the body where the patient is in need of tissue elevation, support, or contouring. Additionally, certain bioadhesives can be used to accomplish similar goals. Surprisingly, embodiments of the methods of the instant invention provide a large, highly noticeable, consistent in degree improvement in facial contour elevation in the cheeks, forehead, brow and jowls using a rapidly-performed small incision method which requires less tissue dissection, less work for the surgeon, less time and less of an ordeal for the patient and less inflammation, smaller post-operative healing time and less scarring. Injectable fixation implants using tissue adhesive administered without surgical incisions but rather simple needle puncture represents another embodiment of the invention. Certain embodiment of the present invention involve altering the anatomic origin, and/or structure of a muscle and enveloping tendon and fascial structure to achieve a surface contour change using specific small incision or even no incision fixation devices.

In one embodiment, the gala or SMAS or other forms of deep or superficial facial connective tissue is fixated to bone by direct placement of a screw and, optionally a compression plate over the screw (which is analogous to a washer), such that the compression plate and screw or bolt are used to create a flush fixation of the soft tissue to the osseous tissue without free suspension of the soft tissue as by a hook or projection device.

The methods described herein afford a very strong soft tissue fixation point, unlike the use of absorbable or non-absorbable hooks or suspension hooks, currently used and advertised by, for example, Coapt Technologies, Inc.

The methods described herein allow for improvements in surgical procedures designed to produce both functional and cosmetic improvements for patients. These improvements include, but are not limited to:
(1) Decreased size of incision resulting in faster recuperation period and faster heal time (reduced post-operative convalescence). Small incision surgery is a preferred surgical method because the potential for unsightly scars or scar hypertrophy is reduced.
(2) Reduction in post-operative edema because of a smaller surgical dissection plan.
(3) Reduction or elimination of the need for intraoperative analgesia-anesthesia because of limited surgical dissection and the capability of producing anesthesia with local anesthesia such as lidocaine or Marcaine with hyaluronidase
(4) A reversible procedure because osseous bolt soft tissue fixation can be removed.
(5) Reduced operative time.
(6) Reduced incision size requiring a minimal amount of suture placements. In one embodiment, the screws of the present invention require an incision size of from about 1 to about 15 mm per screw, preferably from about 1 to about 3 mm per screw.
(7) Direct and immediate assessment of soft tissue placement with most facial muscles dynamic and not paralyzed by excessive placement of local anesthetic or the complete facial paralysis associated with general anesthesia.
(8) Versatility in placement of tissue fixation with tailored fixation possible for different facial configurations and facial contouring as compared to prior procedures.

Certain embodiments of the invention are directed to a surgical method for elevating and/or contouring facial soft tissue in a human patient in need thereof comprising altering or reinforcing attachment of muscle to underlying skull or facial bone by one or both of placing a screw through microincisions or injecting a bioadhesive to the undersurface of said muscle, thereby fixating said muscle and overlying facial soft tissue to said underlying skull or facial bone; wherein said facial soft tissue is elevated and/or a contour, a proportion, dynamic facial wrinkles, static facial wrinkles, and a movement pattern of said patient's face are redefined. In certain embodiments, the screw comprises a compression plate or washer. In certain embodiments, the screw is a self-tapping or self drilling screw. In certain embodiments, the head of said impaled screw is positioned close to flush continuity with the osseous surface. In certain embodiments, the head of said screw is convex or flat. In preferred embodiments, the compression plate is positioned close to flush continuity with the osseous surface. In preferred embodiments, the screw is placed into said underlying skull or facial bone with a screw wrench. In certain embodiments, the skull bones are selected from the group consisting of maxillae, frontal, mandible, nasal, zygomatic and temporal bones. In certain embodiments, screws of the instant invention may be comprised of a material selected from the group consisting of, but not limited to, hard silicon, porous polyethylene, ceramic, steel, titanium, gold, platinum, resorbable poly-L-lactic acid, polyglycolic acid, polymethyl methacrylate, and a composite of these materials

In certain embodiments, the soft tissue is selected from one or more members of the group consisting of facial muscle, gala, SMAS, subcutaneous facial tissue and periosteum.

In certain preferred embodiments, the methods described herein comprise multiple points of fixation.

In certain preferred embodiments, the compression plate is comprised of a material selected from the group consisting of hard silicon, porous polyethylene, ceramic, steel, titanium, gold, platinum, resorbable poly-L-lactic acid, polyglycolic acid, polymethyl methacrylate, and a composite of these materials.

In certain embodiments, the compression plate may be comprised of an absorbable material selected from the group consisting of porous polyethylene, nylon, collagen, cyanoacrylate glue and hyaluronidatev.

In certain preferred embodiments the surgical methods are directed to elevated or contoured facial soft tissue that is the human brow or forehead.

In certain preferred embodiments the surgical methods are directed to elevated or contoured facial soft tissue that is the human midface or cheek region.

In certain embodiments the surgical methods are directed to elevated or contoured facial soft tissue that is the human jowl region.

In certain embodiments the surgical methods are directed to elevated or contoured facial soft tissue that is the human submental region or neck region.

In certain embodiments, the bioadhesive is a cyanoacrylate. In preferred embodiments, the cyanoacrylate or an injection amount thereof is selected on the basis of its inflammatory response in patients. In certain embodiments, the inflammatory response is less than about 5% of patients, preferably less than about 2% of patients, and most preferably less than about 0.5% of patients.

In certain embodiments, the cynaoacrylate is selected from the group consisting of methyl 2-cyanoacrylate, ethyl 2-cyanoacrylate, n-propyl 2-cyanoacrylate, iso-propyl 2-cyanoacrylate, n-butyl 2-cyanoacrylate, iso-butyl 2-cyanoacrylate, hexyl 2-cyanoacrylate, n-octyl 2-cyanoacrylate, 2-octyl 2-cyanoacrylate, 2-methoxyethyl 2-cyanoacrylate, 2-ethoxyethyl 2-cyanoacrylate, 2-propoxyethyl 2-cyanoacrylate, and combinations thereof.

In certain embodiments, the bioadhesive is absorbable, for example absorbable cyanoacrylate.

In certain embodiments, a tip of the injection is in the fibrofatty plane between facial muscle and bone, thereby creating a new muscle to bone adhesion not previously present.

In certain embodiments, the invention is directed to a surgical method for elevating or contouring tissue within the waist region in a patient in need thereof comprising the one or both of placing a screw through microincisions in the subcutaneous connective tissue, fascia, and muscle into an underlying pelvic bone or injecting a bioadhesive to an undersurface of said tissue, thereby fixating said tissue to said underlying pelvic bone; wherein said tissue is elevated and/or contoured.

In certain embodiments, the invention is directed to a surgical method for elevating or contouring soft tissue within the upper arm region in a patient in need thereof comprising one or both of placing a screw through microincisions in the subcutaneous connective tissue, fascia, and muscle into an underlying humerous bone or injecting a bioadhesive to an undersurface of said tissue, thereby fixating said tissue to said underlying humerous bone; wherein said tissue is elevated and/or contoured.

In certain embodiments, the invention is directed to surgical method for elevating and/or contouring tissue within a breast region in a human patient in need thereof comprising one or both of placing a screw through microincisions in the subcutaneous connective tissue, fascia, and muscle into an underlying clavicle bone or injecting a bioadhesive to an undersurface of said tissue, thereby fixating said tissue to said underlying clavicle bone; wherein said tissue is elevated and/or contoured

In certain embodiments, the invention is directed to a surgical method for elevating and/or contouring facial tissue in a human patient afflicted with facial paralysis comprising one or both of placing a screw through microincisions in the subcutaneous connective tissue, fascia, and muscle into an underlying facial, orbital or frontal bones or injecting a bioadhesive to an undersurface of said tissue, thereby fixating said tissue to said underlying facial, orbital or frontal bones; wherein said facial tissue is elevated and/or contoured.

In certain embodiments, the invention is directed to a surgical method for elevating and/or contouring soft tissue in a brow region in a human patient afflicted with blepharospasm comprising one or both of placing a screw through microincisions in the subcutaneous connective tissue, fascia, and muscle into an underlying facial, orbital or frontal bones or injecting a bioadhesive to an undersurface of said tissue, thereby fixating said tissue to said underlying facial, orbital or frontal bones; wherein said soft tissue in a brow region is elevated and/or contoured.

In certain embodiments, the invention is directed to a surgical method for reducing or preventing abnormal facial movement in a human patient in need thereof comprising one or both of placing a screw through microincisions in the subcutaneous connective tissue, fascia, and muscle into an underlying skull or facial bone or injecting a bioadhesive to an undersurface of facial soft tissue, thereby fixating said facial soft tissue to said underlying skull or facial bone; wherein abnormal facial movement is reduced or prevented.

In certain embodiments, the invention is directed to a surgical cosmetic method for reducing or eliminating facial wrinkles or rhytides in a human patient in need thereof comprising one or both of placing a screw through microincisions in the subcutaneous connective tissue, fascia, and muscle into an underlying skull or facial bone or injecting a bioadhesive to an undersurface of facial soft tissue, thereby fixating said facial soft tissue to said underlying skull or facial bone; wherein facial wrinkles or rhytides are reduced or eliminated.

In certain embodiments, the invention is directed to a screwdriver that is attached to an implantable self drilling device for the purpose of compressing soft tissue directly to skull table.

In certain embodiments, the invention is directed to a method comprising fixating a facial muscle to facial bone to decrease contractility of said muscle by immobilizing said muscle and causing a myo-osseous scar to thereby reduce or decrease dynamic facial lines or wrinkles. In certain embodiments, the method is directed to the reduction of facial lines or wrinkles associated with aging. In certain embodiments, said fixation is accomplished with a compression screw impaled through said facial muscle or the surrounding fascia. In certain preferred embodiments, said fixation is accomplished with a combination of a screw and bioadhesive accomplished via puncture or microincision. In certain preferred embodiments, the fixation is accomplished by inducing a scar by applying cautery, a lipolytic agent, radiofrequency, ultrasonic means, or laser energy to induce a scar within the pre-periosteal fat to fixate the undersurface of facial muscle to bone. In certain embodiments, the fixation is accomplished using bioadhesive. In certain embodiments, the bioadhesive comprises polymethyl methyacrylate, cyanoacrylate, a fibrinogen-thrombin combination, albumin gluteraldehyde combination, acrylic, or absorbable cyanoacrylate. In certain embodiments, the dynamic facial lines are crowsfeet, glabellar lines or forehead transverse lines.

In certain embodiments, the invention is directed to a device comprising a metallic or ceramic screw shaft and a compression head, which when applied over the skin or through an incisional site, engages said screw shaft into facial bone to fixate facial muscle and decrease contractility of said muscle and induce submuscular scarring and adhesion of said muscle to facial bone to reduce or decrease static and dynamic facial lines or wrinkles. The device may be used to reduce dynamic facial lines or wrinkles that are crowsfeet, forehead lines or glabellar lines. In certain preferred embodiments, the device may further comprise bioadhesive.

In certain embodiments, the invention is directed to a surgical method for elevating and/or contouring facial soft tissue in a human patient in need thereof comprising altering or reinforcing attachment of muscle to underlying skull or facial bone by injecting a bioadhesive to the undersurface of said muscle, thereby fixating said muscle and overlying facial soft tissue to said underlying skull or facial bone; wherein said facial soft tissue is elevated and/or a contour, a proportion, and a movement pattern of said patient's face are redefined. In certain preferred embodiments, the bioadhesive is selected from the group consisting of fibrinogen, fibrin, thrombin, bovine albumin, collagen, gluteraldehyde, autogenous blood clot, polyethylene spheres, hydroxyappetite, onyx, polyethylene glycol based sealants, cyanoacrolyte, human serum albumin, and combinations thereof. In certain preferred embodiments, said fibrinogen, fibrin, thrombin, bovine albumin, collagen, gluteraldehyde or human serum albumin are recombinantly produced.

In certain embodiments, the invention is directed to a method of compacting and reshaping human muscles comprising steps of: injecting a bioadhesive into a muscle; allowing spreading and curing of said bioadhesive in said muscle; externally manipulating said muscle during bioadhesive curing; creating a synthetic connective matrix within said muscle; and altering a shape and volume of said muscle, thereby improving superficial contour, wrinkle pattern, proportionality, and/or volume of a body region. In certain preferred embodiments the body region is the face, the neck, the buttock, the arm, or the abdomen. In certain embodiments, the bioadhesive is selected from the group consisting of fibrinogen, fibrin, thrombin, bovine albumin, collagen, gluteraldehyde, autogenous blood clot, polyethylene spheres, hydroxyappetite, onyx, polyethylene glycol based sealants, cyanoacrolyte, human serum albumin, and combinations thereof. In certain embodiments, said fibrinogen, fibrin, thrombin, bovine albumin, collagen, gluteraldehyde or human serum albumin are recombinantly produced.

In certain embodiments, the invention is directed to method of altering the viable contractility of muscles to treat movement disorders comprising steps of: injecting a bioadhesive into a muscle; externally manipulating said muscle during bioadhesive curing; accomplishing an internal tethering of fibers within said muscle of fibers within said muscles to each other and to a synthetic connective matrix; and altering a shape and volume of said muscle, thereby decreasing contractility of the muscle to relieve symptoms of the movement disease. In certain embodiments, the movement disease is selected from the group consisting of dystonia, cerebral palsy, painful spasmodic disorder, spasticity, development movement disease, scoliosis, chronic cramping, internal organ smooth muscle spasms, bladder spasticity, external bladder spasm associated with prostate hypertrophy, intestinal spasms, esophageal spasms, and stomach spasms. In certain embodiments, the bioadhesive is selected from the group consisting of fibrinogen, fibrin, thrombin, bovine albumin, collagen, gluteraldehyde, autogenous blood clot, polyethylene spheres, hydroxyappetite, onyx, polyethylene glycol based sealants, cyanoacrolyte, human serum albumin, and combinations thereof. In certain embodiments, the fibrinogen, fibrin, thrombin, bovine albumin, collagen, gluteraldehyde or human serum albumin are recombinantly produced.

In certain embodiments, the invention is directed to a method of altering the viable contractility muscles to alter a surface contour or wrinkle pattern of a human body region comprising steps of: injecting a thermal cautery into the undersurface of a muscle; effecting fat coagulations and an inflammatory bioadhesion between muscle, bone, or muscle to muscle; and altering a shape and volume of said muscle, thereby decreasing contractility of the muscle to alter a surface contour or wrinkle pattern.

In any of the embodiments involving injection of bioadhesive, the invention contemplates repeated injections.

### DESCRIPTION OF THE FIGURES

FIG. 1: Typical approach to face lift using skin resection and SMAS tightening. The conventional facelift requires deep and superficial planes of dissection which are inherently more traumatic causing a greater degree of postoperative inflammation, and the potential for injury to critical vascular and nervous structures. Facial nerve injury is a serious complication of the conventional facelift.

FIG. 2: Diagram of the human skull. Red indicates facial muscle attachment points to the skull. Facial muscle is part of the SMAS, facial muscle is in turn attached to the deep skin.

FIG. 3: Improvement in facial contouring and cosmetic facial appearance. Patient is a 72 year old female with a history of parotid cancer. Patient underwent radical resection 20 years before pictures were taken. In order to cure her cancer, she needed to have the entire facial nerve removed on the right side. Subsequently, the face became paralyzed with complete absence of facial tone leading to disfiguring brow ptosis (drooping) on the right side (FIG. 3a). Patient underwent three implantable bone soft tissue compression implants on the right side of the brow fixating the brow and its atrophied muscle to the bone (FIGS. 3b and 3c). FIGS. 3b and 3c demonstrate the technique of compression implantation and the device, with impalement of soft tissue, SMAS (with orbicularis and frontal muscles in this example), and compressing this soft tissue later. Cosmetic facial appearance was felt to be markedly improved by the patient, her family and the surgeon (FIGS. 3d and 3e).

FIG. 4: Treatment of progressive involuntary facial movement disease (blepharospasm)—improvement of facial contouring and cosmetic appearance. Patient is a 69 year old engineer who was diagnosed with progressive involuntary facial movement disease (blepharospasm) causing involuntary eyelid closure and loss of functional vision (FIG. 4a). Medicinal therapy with multiple agents were deemed to be failing by the patient, his family and his ophthalmologist. He underwent pinning of the SMAS and frontal muscles as well as orbicularis which resulted in elevation of the brow, alteration in the insertion of the orbicularis muscle, redirection of muscle fibers, and dramatic improvement in his ability to control the involuntary movement causing eyelid closure. Brow elevation is seen two weeks post operatively (FIG. 4b).

FIG. 5: Treatment of involuntary blepharospasm—improvement in cosmetic appearance and facial contouring. Patient is similar to the patient in FIG. 4 with involuntary blepharospasm who underwent implantable soft tissue compression bolts in three locations FIG. 5a. FIG. 5b is her appearance one month post implantation showing substantial elevation of the brow and improvements in facial contour

FIG. 6: Treatment of paralyzed eyelids for improved cosmetic appearance and facial contouring. Patient is a person with paralyzed eyelids (FIG. 6a) who underwent fixation of the lateral orbicular and surrounding connective tissues. This caused tightening of the lateral canthal tendon and elevation of the eyelid which improved cosmetic appearance facial contouring. The procedure also increased the functional closure which was accomplished by a second weight implanted in the upper eyelid (FIGS. 6b, 6c and 6d).

FIG. 7: Sagittal view of the human face trasecting the eye. The white configuration under the skin represents fibrofatty tissue. The fibrofatty tissue is seen anterior and posterior to facial muscles and below the eye. The figure illustrates the extent of the pre-periosteal, subaponeurotic and sub dermal fat can be seen on the magnetic resonance image of the forehead scalp and skull table. The T1 weighted image demonstrates the high contrast fat layer in the pre-periosteal, sub-aponeurotic region and in the anterior region of the orbicularis frontalis muscle region above the eyebrow. The fat pads in front and behind the muscle (lighting up white in the scan) represent a low resistance “sandwich” by which these facial muscles move without substantial resistance. Increasing friction within these fat planes by fixation or scarring alters tone and contractility of the muscle leading to surface changes and changes in functional performance

FIG. 8: Map of the human facial anatomy to identify facial muscles and enveloping fascia subject to the micro incision screw or glue injection.

FIG. 9: Demonstrates the vast area of facial bone not containing an origin of a facial muscle or a critical nerve or vessels at risk from the screw placements.

FIG. 10: Displays muscles that could be subject to fixation, redirecting support of the facial muscular mask

FIG. 11: Displays endomysium of a muscle.

FIG. 12: Displays areas where glue can influence intermuscular adhesion and intramuscular cohesiveness to effect contour and surface changes on the human head and neck region.

FIG. 13: This patient underwent treatment for blepharsopasm using a myo-osseous fixation procedure. Unlike prior procedures the fixation was accomplished with a cyanoacrylate glue (2-Octyl cyanoacrylate). Post operatively blpeharsoapsm was 90% improved in the first week. Lateral orbicularis muscles fixation accomplished with tissue glue was excellent and functioned in lieu of an mechanical fixation device to tether muscle to bone and effect improvement of involuntary eyelid closure. Serendipitously, the crowsfeet wrinkle pattern usually present in this location was significantly improved.

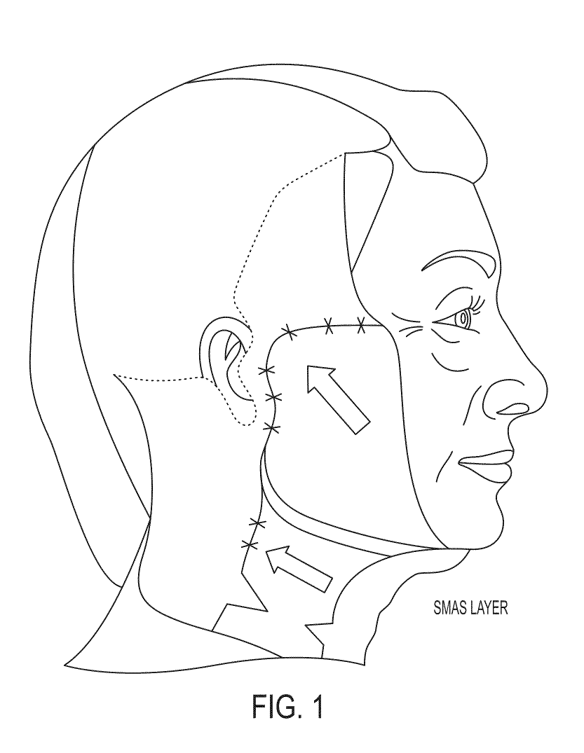

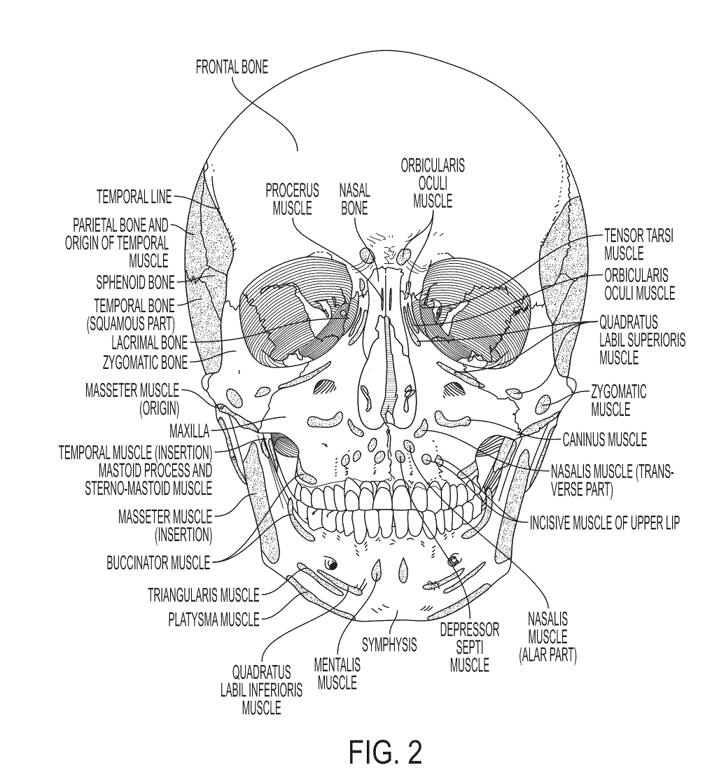

### DETAILED DESCRIPTION

As used herein “osseous tissue” means bone tissue, the major structural and supportive connective tissue of the body. Osseous tissue forms the rigid part of the bone organs that make up the skeletal system.

As used herein the “superficial muscular aponeurotic system” or (SMAS) refers to an area of musculature of the face. This muscular system is manipulated during facial cosmetic surgery, especially rhytidectomy.

As used herein, a “self-tapping or self drilling screw” is a screw that has the ability to advance when turned, while creating its own thread. Self-tapping or self drilling screws are commonly used with sheet metal and plastic components. This ability is created sometimes by having a gap in the continuity of the thread on the screw. These edges can cut their own threads as the screw is driven into the material, usually wood or plastic. Self tapping or self drilling or self drilling screws also exist for metal. They function by having a cutting edge which drills away the material, making a hole for the screw to go into. Self tapping or self drilling screws are most useful when doing metal and wood work.

As used herein, a “compression plate” is a surface that squeezes, compresses or pushes layers of soft tissue directly against bone to thereby cause fixation of the tissue, inclusive of muscular tissue, and alters the muscular tissue's relation to a low-friction perimuscular environment.

As used herein, the term “flush continuity with the osseous surface” means the screw or compression plate is placed tightly against bone such that the degree of palpable ridge from the skin surface is minimized.

As used herein, “bioadhesive” is any liquid or gel which when placed in a plane between muscles and bone causes adhesion. This allows a plastic surgeon to remodel the soft tissue to bone using free movement of the muscle overlying the bone facilitated by the low resistance fibro fatty plane underlying the muscle. A bioadhesive is able to be injected under muscle. This allows adherence of muscle to the bone structure which in turn alters the origin of the muscle, functional contractility of the muscle, and support of overlying soft tissues generated by muscle tone and support.

As used herein, “low resistance plane” or “low friction plane” is the fibro fatty plane between bone and the undersurface of a muscle overlying the bone.

As used herein, “skin puncture” is a puncture through skin made with a medical needle between 4-32 gauge. In a preferred embodiment, a skin puncture is made with a medical needle between 25-30 gauge.

As used herein, “small incision” is an incision less than 10 mm. In a preferred embodiment of the present invention, a small incision is less than 3 mm.

As used herein, “large incision” is an incision greater than 1 cm.

As used herein, “external manipulation after bioadhesive injection” refers to movement of soft tissues (or muscle) over bone, facilitated by the low resistance fibrofatty tissue plane underlying the muscle, which is performed after injection of a bioadhesive. This allows binding of the undersurface of the muscle to bone thereby creating an alteration of surface soft tissues with respect to proportion, contour, function, skin wrinkle pattern, elevation, or other configuration desired by patient or surgeon.

As used herein, a “method of injection as to avoid nerves, arteries, and veins” refers to anatomic knowledge possessed by the surgeon, which contemplates natural variations of usual and unusual positions of critical nerves and vessels which can be injured by microincisions, implantable devices, or injections of bioadhesives.

As used herein, “body proportionality” refers to size or volume of a body region relative to another body region. For instance, upper cheek size relative to lower cheek size, upper neck size relative to lower neck size, and buttock size relative to thigh size, and so on.

As used herein “muscle compacting” or “muscle reshaping” by bioadhesive, refers to the process of injecting a muscle with a bioadhesive and shaping the muscle to alter the contour of the face or other body part by external manipulation, by which the injected bioadhesive functions as a connective synthetic matrix replacing the lax collagen forming endomysium, perimysium, and epimysium.

As used herein, “use of needle based thermal cautery to achieve undersurface muscle fixation” refers to another approach to cause fixation of muscle to bone. The method involves application of cautery via fine needle to the undersurface of the muscle, which results in fat deletion and contraction followed by inflammation and scarring in deep facial layers, or other body part layers. The result is an adhesion endogeneously created by the body, which results in adhesion and alteration in surface configuration. The cautery can be unipolar microbipolar, or battery powered device which transmitted energy through a puncture site to the undersurface of the muscle which created a adhesive scare. This method may be used singly or in conjunction with screws or bioadhesive methods as described elsewhere in this disclosure.

As used herein, the “use of lipolytic agents to achieve bioadhesion” refers to using lipolytic agents to affect the functional components and contribution of the surrounding fat layers to muscular contractile effect. These agents cause lipolysis, and can be used to increase resistant within surrounding muscle tissues and increase the resistance to muscular contractility. Such agents include bile acids or bile salts or other such agents which achieve lipolysis Use of physical energy systems such as radiofrequency or ultrasonic devices can also be used to dissolve fat cell membranes. This diminishes the fatty layers surrounding the muscles and thereby increasing resistance against muscle action. Other physical agents such as laser energy applied via endoscope, or needle applied radiofrequency and the like can achieve a similar effect.

As used herein, a “synthetic connective matrix” refers to bioadhesives injected into a muscle that insinuates and spreads when cured to form a “compaction” and/or internal fiber tethering, “internal restriction” of the muscle, decreased volume, and decrease contractility of the muscle.

As used herein, the “origin” of a muscle is the point at which it attaches to a bone or another muscle. The structure that the origin is attached to is not moved by contraction of the muscle. The opposite end of the muscle is the insertion.

As used herein, the “insertion” of a muscle is the point at which it attaches to the skin, a bone, or another muscle. The insertion attaches the structure that will be moved by the contraction of the muscle. The opposite end of the muscle is the origin.

As used herein, “altered origin or insertion” of a muscle is creating an adhesion of muscle in area, for example in an area of bone, where normally no adhesion is present.

The screw plate screw and the compression fixation plate can involve the use of tapping or self-tapping or self drilling or self drilling screws that are placed in a strategic area in such a fashion that a facial plane is compressed. The facial plane could include the gala or the SMAS or other deep facial or superficial facial plane.

The gala, if used, is fixated to the skull over the frontal bone medially or laterally to achieve brow elevation, or over the zygomatic bone to achieve mid face and lower mid face elevation, and in regions of the zygoma or the mandible and jowl elevation. Such screws are designed so that full penetration to the skull over the neurocranium does not occur. In one embodiment, the length of such screws can vary from between about 0.25 mm to about 10 mm, and preferably from about 2 to about 8 mm for skull and facial bones. In another embodiment, the length of such screws can vary between from about 1 to about 50 mm, depending on the body region of interest. The selective advantage of this approach allows small incisional surgery without the use of extensive surface dissection which facilitates rapid wound healing and reduces the procedures to being minimally invasive, as opposed to more open requiring larger wounds and more extensive delays in wound healing and the potential for greater scarring and disfigurement post operatively.

The screw is customized to the portion of the skull being used. Thicknesses of the skull are determined anatomically and also with neuroradiography using computerized axial tomography for design. The screws are placed with a fixation wrench after a small incision is made, and adequate mobilization of the subcutaneous connective tissues are accomplished. This can be done with the patient awake, unlike former procedures for brow lifts in which the patients are often under general anesthesia. The fixation points are marked off and local anesthesia is used with diffusion enhancing agents such as hyaluronidase. Alternative forms of anesthesia involving regional nerve blocks are also possible to limit soft tissue distortion so that the osseous fixation can be more accurately accomplished. Once anesthesia is achieved of the soft tissue and bone, the facial plane is dissected and a small buttonhole is placed through the facial plane. The facial plane is then pulled to a desirous location and the bolt is placed into the bone, essentially lifting soft tissues below. In the case of brow lift, this is done in a fashion to achieve both accurate elevation and contouring of the brow. In the case of lower mid face, the placement is over the zygoma, fixating the suborbicularis fascia to points of fixation on the lateral orbital rim over the zygoma. The compression plate used is a conventional washer over a screw, to achieve a large platform fixation over bone to further enhance the strength of the elevation and fixation point. Screws may be absorbing or non absorbing materials. Preferred materials include titanium, stainless steel, or absorbable material such as chromic, nylon, polymethylmethacrylate, or hardened forms of silicone. Glue arrangements may be arranged with various forms of epoxy to further enhance fixation of the screw's endplate. In other words, the screw and/or compression plate may be used in combination with a glue or adhesive. The glue may not only cause an increase in tensile strength of the screw's attachment to the bone, but also may cause adherence of the undersurface of the facial or targeted muscle to the bone. It is important to note that in order to facilitate natural contour and feel over a healed wound, the screws are placed flush with the skull so raised elevations are minimally perceived by the patient, or any potential observer.

Advantages of such approaches include a small incision or needle puncture incision with minimal suture closure and minimal surface scarring.

Diameter of screws can vary from between about 0.25 mm to about 4 mm, depending on the fixation point. The head of the screw can vary from between about 0.25 mm to about 10 mm in diameter.

Said procedures are done with full knowledge of facial motor and sensory neural positions. Motor and sensory neural positions can be localized with nerve stimulators, Doppler devices for vascular bundle nerve identification, and general knowledge of anatomic variation.

The screw washer plate can vary in size from about 2 mm to about 25 min in diameter, preferably between about 2 mm to about 10 mm in diameter. It can also have various geometric forms, for example circular or circular with flush projecting platforms. Here again, it is important that there is no direct extension of soft tissue over the fixation plate, but rather a flush bolting of soft tissue to skull or gluing the undersurface of muscle to the skull, for example by way of bioadhesive.

Screw head configurations can be philips, linear or any geometric configuration which enhances mechanical integration to the driver. The driver must be able to fixation the screw head in such a fashion to allow impalement of the facial plane to be fixated so that the deep or superficial facial planes can be elevated and fixated along a higher position on the facial bones.

As the natural aging process involves the descent of soft tissues, the purpose of the bolting screws or glue adhesives described herein is to create a suspension of soft tissue back on the position of the skull in which soft tissue has fallen. By virtue of doing so, the contour of the surface is changed, remodeled, reshaped and improved to a more juvenile appearance. Hyperplacement of soft tissue is anticipated with compensatory falling in a postoperative period.

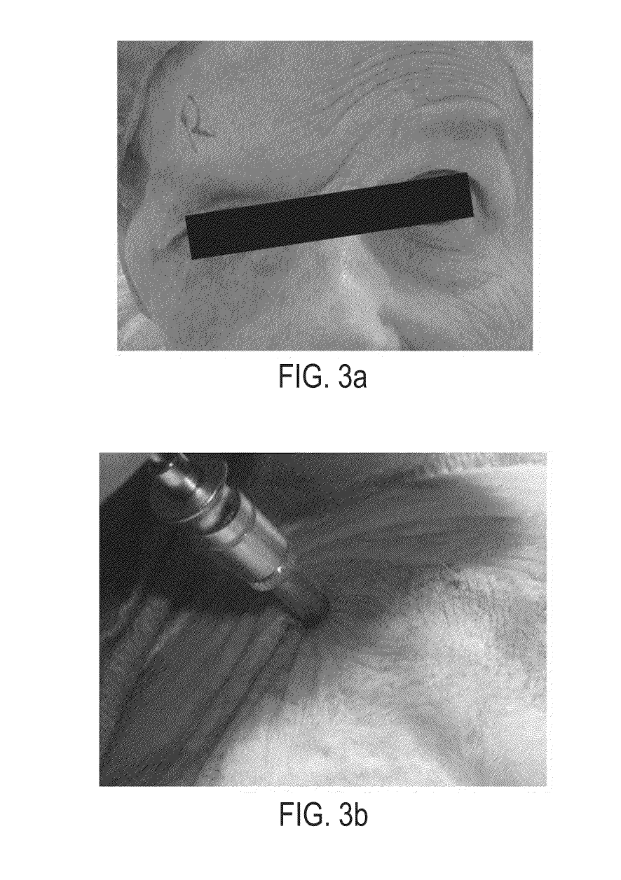

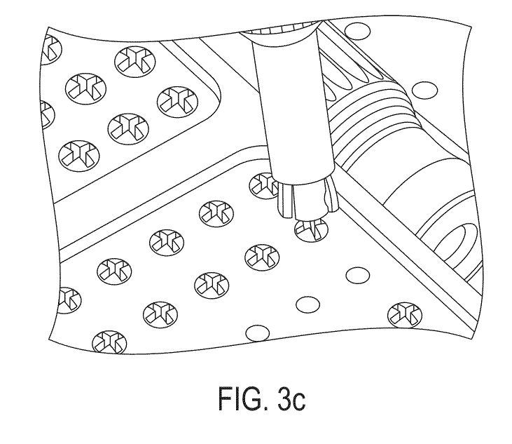

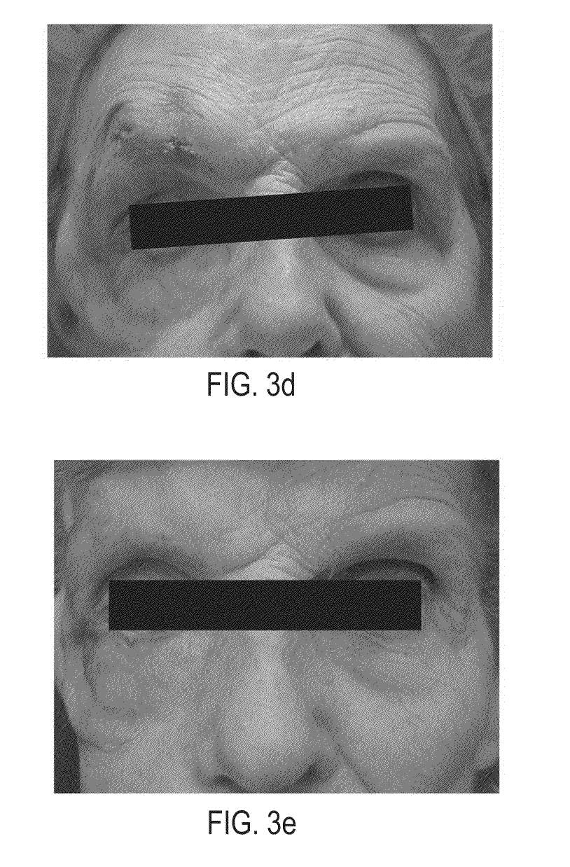

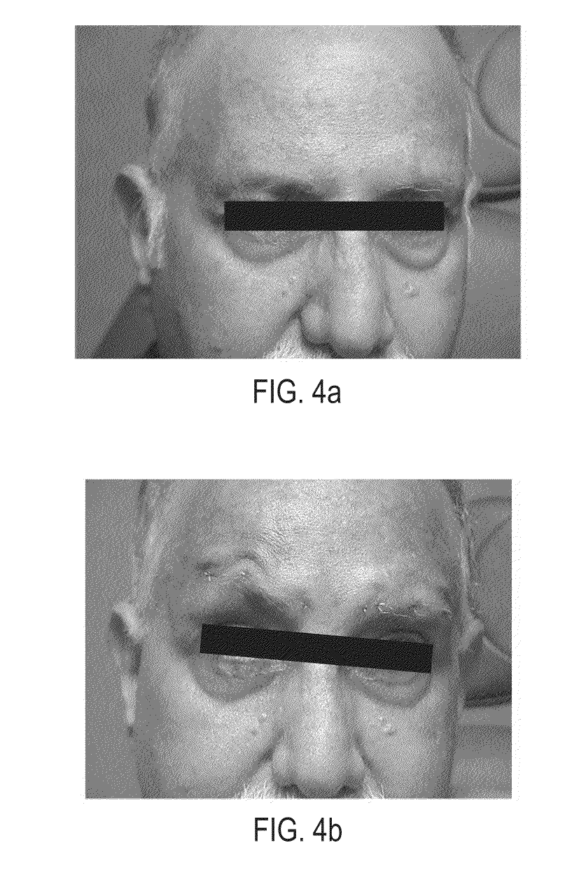

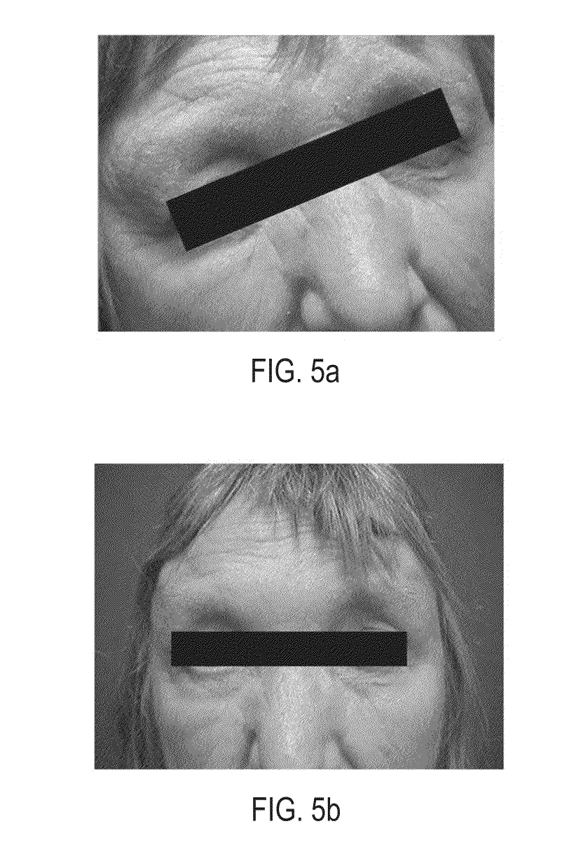

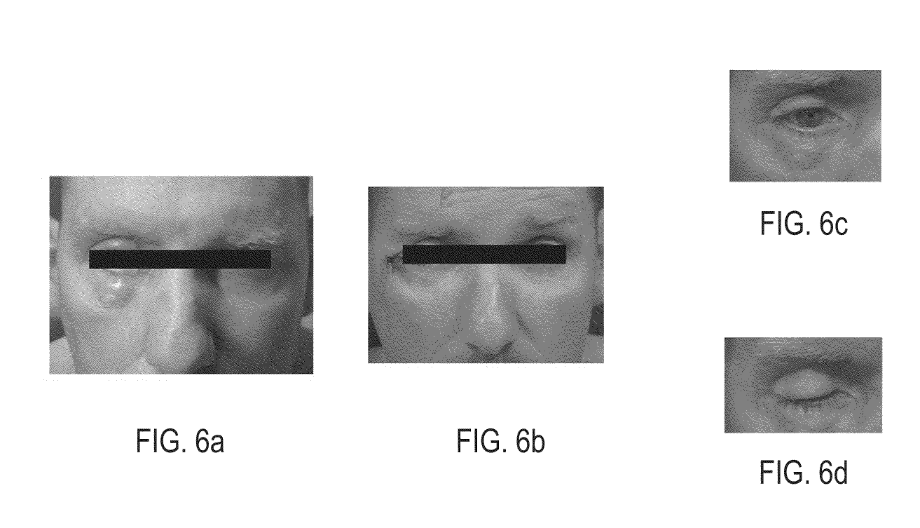

A diagram of such a screw arrangement and prototype is given in FIG. 3c. This diagram should not be limiting to other configurations in which there is a screw and fixation plate arrangement. Selective advantages of a fixation plate screw method include:
1. Small incision.
2. Direct capability to do the procedure under local anesthesia.
3. Increased strength of fixation point over previous methods of fixation such as purse string sutures, tissue resection, or other forms of soft tissue suspensions with ligatures.
4. Limited surgical dissection with increased rate of wound healing postoperatively.
5. Technical simplicity in performing with increased speed of procedure.
6. Capability to perform procedures on an outpatient basis without use of a major operating room.
7. Greater chance of permanent effect.

### Concept of Alteration of the Origin and Insertion of Muscles Using External Bolting to Osseous Structures

The use of subcutaneous fixation self-tapping or self drilling or self drilling screws and muscles and soft tissue compression into osseous structures effectively can be used to change the origin and insertion of muscles. Muscles produce mammalian movement via attachment to various bone providing stability and direction to forces created by muscle contraction. By externally changing the fixation of muscles on bony platforms, it is possible to alter the vector forces generated by muscular contraction causing alteration in the type, direction and intensity of movement elicited by such contractions.

As an example, blepharospasm patients suffer from involuntary contractions of the orbicularis muscles. The orbicularis muscle has three basic segments: pre-tarsal, pre-septal, and pre-orbital. The pre-orbital section is the most well developed, eliciting the most forceful contraction on stimulation. The origin and insertion of this segment of this muscle are the medial canthal ligaments and adjacent bone. When the titantium bolt is placed elevating the brow, an alteration of the insertion and origin of the muscle is created such that
(1) Resting tension on the muscle may be changed
(2) Forces generated during contraction are re-directed in different vectors, mitigating the protraction (closure) of the eyelid
(3) Force generated by the muscle may be reduced
(4) Proprioceptive sensory feedback to the central nervous system may be changed such that involuntary movements are mitigated

Redistribution of muscular attachments can effectively change fiber direction and the extent to which fibers may contract.

In the case of the orbicularis muscle, fibers in the pre-orbital segment are oriented in a circular fashion except at medial points of attachment to tendon and bone. The titanium peg, or screw, or bolt or self-tapping or self drilling or self drilling screw, or compression plate or bioadhesive, re-orients the fibers into a direction such that the fibers are no longer circular in orientation but rather, obliquely configured as well as fixed to the undersurface of the bone, thereby producing immobilization of the fibers. This change in orientation and configuration depresses the contractility of the muscle, thereby altering function as well as surface appearance of the region. This arrangement also increases the frictional surface under the muscle to thereby restrict muscle contraction and increase inertia to movement.

Elevating the brow further functions to fixate the orbicularis to the frontal bone along the superior orbital rim both medially and laterally. The orbicularis muscle is a sphincter muscle that works by closing space in the middle. The effect of fixating the orbicularis muscle is to increase resting tension on the muscle fibers much like pulling on the inside of an elastic band. This fixation of the orbicularis muscle prevents sphincter muscle contraction. The resting contraction of the circular loop of muscle fibers, redirecting the angle of muscle fibers so that the vector forces are re-angulated causing the forces caused by contraction of muscle to have a reduced effectiveness (reduced mechanical advantage) therefore reducing the symptoms caused by the involuntary muscle contraction. In other circumstances, providing a new origin or insertion may render weak contractions more effective by increasing the tension and simulating the direction of the muscle fibers causing a reduced force of contraction to be more effective in movement. Adherence of the undersurface of the facial muscle can serve to reduce its tone once a firm adherence occurs and the frictional relationship between the bone and the attached muscle is increased.

### Screwplate, Pin, Screwhead Fixation Device

The method and technique require use of a sharply tapered screw or pin capable of penetrating both soft tissue and bone structure without undue trauma to surrounding tissues. In order for the surgeon to accomplish angulation of penetration or fascial-muscle snaring hooking and penetration, firm fixation of the compression pin, screw head, or screw plate is necessary. This allows adequate control of placement which is critical to appropriate soft tissue osseous integration and fixation which drives the change in surface structure elevation and contour changes.

The driver must firmly grasp the screw head via a circular holding device which is adequately tapered to fixate the screwhead and attach the head to the driver. Such devices, which may be referred to as “screw wrenches,” “fixation devices,” or “fixation wrenches” herein are conventionally engineered to the placement of screws to fixed fracture plates, however such devices are not conventionally used for tissue snaring, looping or impaling. Screw driver wrenches used for bone plates for fracture repair can be adapted to achieve this soft tissue function via a micro-incision to snare, impale and ultimately fixate connective tissue directly into the bone structures under a compression plate. The screw wrench can resist lateral torque produced by the surgeon's hands and therefore can hold the screw in tight portion allowing directional control. The screw wrench can secure a screw at different angulations. Analogy is made between the screw wrench and screw to the surgeon's needle holder and needle. The needle must be held with precise control and fixation in order to avoid impaling or otherwise hurting, for example, arteries, veins, eyes and the like. In the case of screws, the screw head must be able to resist substantial torques and forces, for example substantial lateral torques on the screw head. The screw cannot be allowed to “tiddlywink” away from the surgeon's desired placement. It is contemplated that repeated adjustments of the screws may be possible or necessary. An example of a fixation system is given in FIG. 3c.

### Components of the Implantable Fixation Device

The implantable device consists of a compression plate (screw head with and without washer ring), and drilling-fixation shaft for soft tissue stabilization. The drilling shaft may vary from 1 mm to 50 mm, preferably 2 mm to 8 mm when used on the facial or skull bones. The diameter of the drill shaft may vary from 0.2 mm to 10 mm, but is preferably 1 mm to 3 mm. The compression plate may substantially vary in size and configuration, however it is tailored to the anatomic region where the implantable device is positioned. Compression plates can vary between 3 cm to 1 mm.

### Mitigation of Complications

Depth of penetration into the skeletal structure may result in unnecessary pain and risk of the insertion and soft tissue fixation procedure. Use of screw lengths appropriate to the anatomic thickness and position of the bone is critical in preventing excessive penetration of the fixation shaft of the compression screw and plate to the bone, excessive penetration may:
(1) be associated with excessive pain
(2) cause damage to deep vascular structures and initiate hemorrhage
(3) represent a risk of disruption of intracranial vessels if transcranial penetration occurs during facial application

The use of computerized imaging devices using computerized axial tomography or other forms of imaging digital data devices to achieve intra-operative anatomic registration is helpful in confirming depth of penetration of the fixation shaft and optimum placement of the drill shaft into bone. Optimum fixation requires placement of the shaft into bone of appropriately matched thickness relative to the length of the fixation shaft, avoidance of sinus cavities which, if penetrated, can destabilize soft tissue fixation and partial thickness penetration to avoid damage to deeper structures (such as brain and meningeal coverings for forehead and facial fixations). Use of computerized imaging devices may be useful in individual assessment of bone thickness which can substantially vary among individuals.

### Attachments of Facial Muscles to the Facial Bones and the Aging Process

The facial muscles are contained within the superficial muscular aponeurotic system as has been described above. With aging, the connective tissue (collagen) component of this tissue becomes increasingly lax resulting in diminished support of soft tissues associated with sagging. Tissues may also intrinsically age with fat redistribution, muscle atrophy, bone remodeling and dermal laxity. An age related change often not cited on the human face is integrity and strength of deep facial muscle attachment to the facial and skull bone. These tissue origins essentially hold the highly motile SMAS to the bone lending support to facial movement and resting facial position.

The implantable devices described herein provide a method for increased facial muscular support which leads to alterations in facial contour and surface configuration in a more youthful and vital direction. Reinforcement of natural attachments (muscle origins) results in more youthful and functional performance of facial muscles, as well as other muscle groups targeted for attachment reinforcement. Natural critical attachments can be studied for optimum placement of implantable devices.

### Bioadhesives

In another embodiment of the present invention, an alternate muscle to bone periosteum adhesion and muscle to muscle adhesion is accomplished with an implantable or injectable bioadhesive or tissue glue. This method may use concepts described throughout the instant specification with respect to screws and/or compression plates; however, the use of bioadhesives improves the administration technique from microincision to mere skin puncture. This leaves no appreciable scar. In one embodiment, an injectable material, preferably comprising a substantially inert material with quick drying dynamic is used. This injectable material is capable of sustaining a strong adhesion between the undersurface of facial muscle and underlying bony structures. This allows for fixation of the muscle and support. This muscle fixation is useful to control involuntary movements and also to allow for support of soft tissue to restore a youthful and aesthetically original contour surface structure to the human face. In one embodiment, the method reduces or eliminates wrinkle patterns during dynamic facial movements.

In one embodiment, injectable materials include bioadhesives consisting of cyanoacrylate derivatives, such as 2-Octyl Cyanoacrylate, fibrinogen-thrombin combinations, gluteraldyhyde based glues, dental crown cement fixation glues, and inflammatory provoking adhesives which may allow for a small inflammatory response causing a collagen “scar” based adhesion. In one embodiment, an injection device comprises a needle, a single or multiple chamber injections system, or a multiple needle system in order to inject the glue in multiple sites. On the human face, targeted areas would include placing the glue over the surface of the frontal bone, zygomatic and lateral orbital bone, face of the maxillae, and rim anterior or posterior surface of the mandible. As every human face pattern is different in the aesthetic application, emphasis on certain areas would be considered on a case by case basis. The amount of glue injected should be limited by inflammatory response, surface bumps or bulging, and anatomic consideration to avoid nerves, arteries and veins. Multiple injection sessions over a defined period of time can limit the risk of uncontrolled spread of the glue and excessive inflammatory response, and to adjust adhesion strength. It is anticipated that dispensing of the glue shall be accomplished with unit injectable quantities associated with single or multiple use needles. Each injection quantity would have been predetermined to limit the degree of inflammatory reaction so not to promote excessive scar formation visible on the skin surface.

In certain embodiments, adhesion is injected via a plastic or metallic needle so that the needle lumen is placed under the muscle within a mobile fat plane, so that the adhesive can be delivered through a puncture site (non incisional), and the adhesive injected with immediate surface elevation by the hand or direction of the physician or clinician, so that the underlying muscle within the mobile tissue plane containing the hardening glue becomes adherent to facial bone-periosteum causing an altered origin, that is fixation point (support point) so that a soft tissue facelift can be achieved, without the need for an incision. It is anticipated that multiple injections of the tissue adhesive at the time of applications and over a period of time may be needed.

The embodiments herein described altering muscle and soft tissue attachment to bone to achieve change a surface contour of the face and other body regions without use of conventional large incisions.

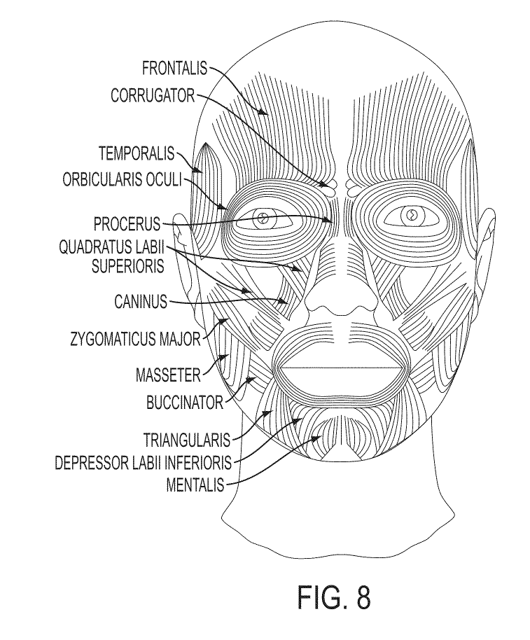

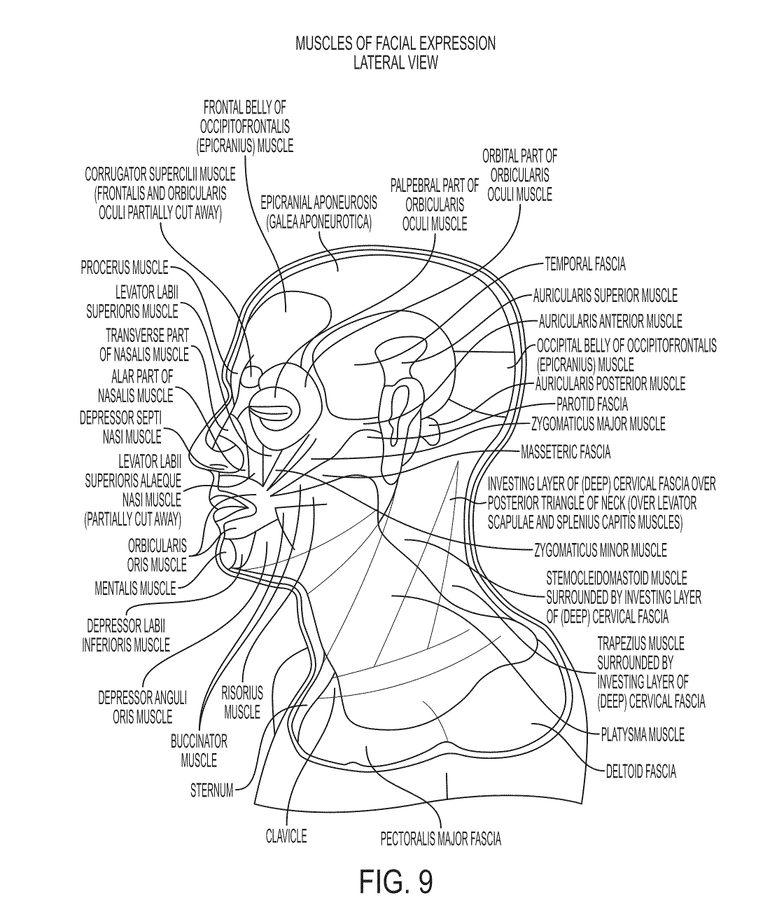

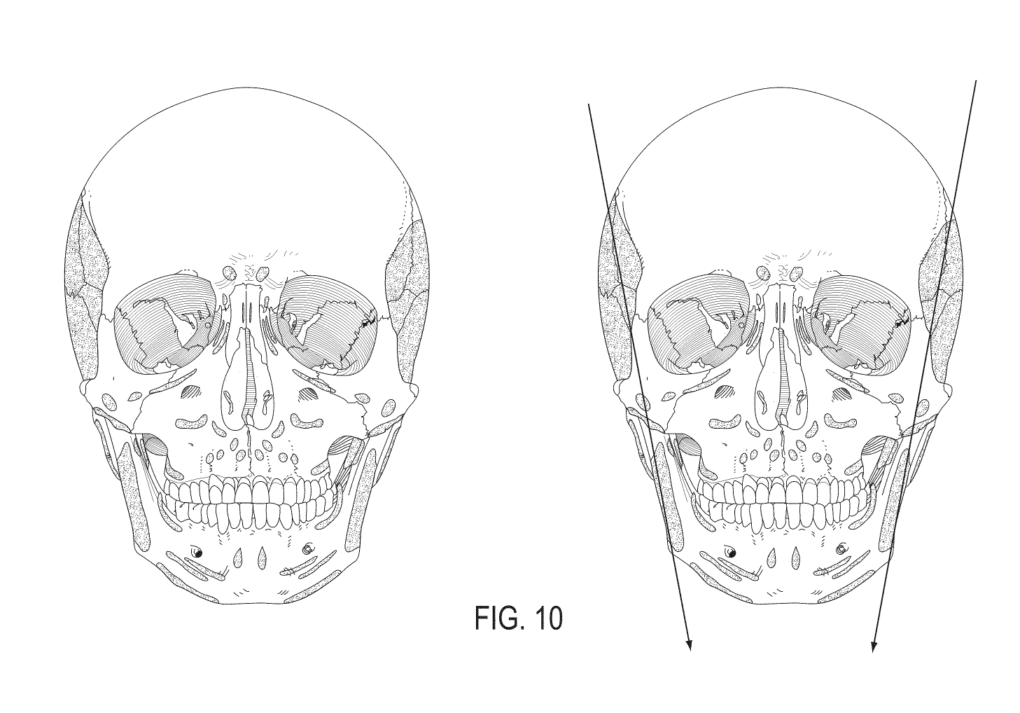

A map of the human facial anatomy is given in FIG. 8 to identify facial muscles and enveloping fascia subject to the micro incision screw or glue injection. FIG. 9 demonstrates the vast area of facial bone not containing an origin of a facial muscle or a critical nerve or vessels at risk from the screw placements. In FIG. 9, any of the listed muscles could be subject to fixation, redirecting support of the facial muscular mask (see FIG. 10). These muscles with attachments and enveloping fascia comprise the foundation for the individual contour, movement and wrinkle characteristic and proportionality of the human face. Arrows on FIG. 10 display the characteristic taper or V-shape of the human skull and face. The overlying soft tissue tends to sag as humans age, thereby distorting the natural shape and proportionality of the human face. Methods and devices described herein can correct or counteract this distortion.

### Screw Size

Screw sizes can vary as well as screw head designs. Dome screw head, convex screw head, flat screw heads, and screw heads with expandable compression heads are all possible types of the implant designs. The ideal screw head would be the most flush to flat and least palpable after surgical implantation. Screw head may vary considerably from small puncture incisions to large compression plates. Compression plats may be washer shaped or linear shaped and implanted via side punctures so that screw penetrates compression plates at one or more orifices. The compression plate may be absorbable or made of a not absorbing metal, acrylic, polymethyl methacrylate or silicone. The screw hand may contain microhooks, serrated surface to enhance anterior or superficial tissue hold. In one embodiment of the invention, the screw are small enough that no superficial suturing is needed. Screw may be used with various forms of glue. The current practice of soft tissue fixation to bone involves and predrilled cylindric hole with a plug in absorbing hooked device. This system is flimsy and offers less support than a drilled penetration into the outer table of skull flat bone.

### Anatomic Plane Targeted for Glue or Device Placement and Method of Delivery

As the intent of the minimally invasive procedure described herein is to provide a reinforcement of the origin and origin/insertion relationships between facial muscles and bone, the correct anatomic plane of the injection must include the fibro fatty plane between the undersurface of the facial muscle or other anatomically defined muscles and the underlying bone covered by periosteum. This plane should be fixated by either injectable glue, placement of compression screw or fixated by adhesion based implant for the purpose of altering contractility of muscle, and/or altering facial contour, and/or influencing wrinkle patterns of skin during dynamic muscular activity. The optimal method is least invasive, requiring the smallest incision. In one embodiment, the incision is merely a puncture site. Any anatomic plane between bone and muscle represents an important target for reducing muscular activity by increasing friction, inducing tethering, and functional resistance to the internal forces produced by the muscle itself. Internal muscular activity is conventionally defined by neuromuscular relationships, muscle mass, and effective chemical reaction between actin and myosin proteins.

Bioadhesives or Glues Applicable for Submuscular and Intramuscular injection

The following represent non-limiting exemplary bioadhesive or glue types which can be used in various embodiments as an injectable to achieve myo-oseous fixation to control contour changes, wrinkle pattern reduction, and/or elevation of the human face via injection These agents are qualified by adhesive properties, inflammatory response and duration of adhesion. Although variations are possible based on dose and position, the overriding principle of myo-osseous fixation can be accomplished by one or more of these agents, at varying doses dependent on strength of adhesion, inflammatory complications and duration of effect. Other qualifying parameters include coloration quality with clear or lighter cured glue color being preferable, flow characteristic via delivery device, curing rate (hardening rate), viscosity, and cohesiveness to prevent migration or possible embolic complications. Bioadhesives or glue may be administered in conjunction with metallic screws or other types of fixation devices which facilitates reconfiguration of attachments of the undersurface of the facial muscles to bone. External devices such as temporary bandages, masks, or tapes can also be used to assist in this minimally invasive procedure.

### Fibrin-Thrombin-Fibrinogen

This glue type consists of a duo delivery system which involves the injection of fibrinogen followed by thrombin which catalyzes the conversion of fibrinogen to monomers which cross link to form the fibrin clot. The fibrinogen is often formulated in higher concentrations than the human plasma and can be derived from pooled plasma or from recombinant origins. Thrombin also can be derived from pooled human blood products or from recombinant manufacturing technology. Plasminogen may be removed from such products as this agent when activated may causes fibrinolysis which degrades the clot and seal properties. Alternatively, agents which inhibit plasmin which can further limit clot degradation. One such inhibiot is aprotinin which is used in the commercial product Tisseel™. Recombinant blood based products limit potential contamination with hepatitis B, C and HIV as well as prion based disease.

It is anticipated that glue delivery is needed in multiple locations and given serially to achieve a lasting seal and bioadhesion.

Fibrin based sealant have the potential to induce growth factor for fibrocytes which can enhance sealing capability over time. These materials can control bleeding which can be beneficial when injected into a vascularized muscle.

Commercially available products include Tisseel, Evicel, Vitage, and Cryoseal

### Thrombin

Thrombin may be used individually as a glue and binding agent and may be used in its recombinant form or native form from human or animal based donors. This agent may be used with gelatin.

### Polyethylene Glycol Based Sealants and Glue

Currently Duraseal™ and CoSeal™ are available in the US. These agents can be used to control bleeding and have sealant and binding capability which may be adapted to muscle bone fixation. Durseal is commonly used in the head for outer brain covering procedures.

### Gluteraldehyde-Albumin Sealants

These sealant consist of purified albumin with gluteraldehyde which when mixed forms a covalent bond between the matrix and cell surfaces. BioGlue™ is a non-limiting example.

Cyanoacrylate based Sealants

Non-limiting examples are 2-octyl cyanoacrylate, N-butyl 2 cyanoacrylate, and absorbable cyanoacrylates, known under the trademark OMNEX™. These agent can effectively stop bleeding. Binding strength is better than fibrin based sealants. Low volume of agent can be effective. Inflammatory potential can potentially enhance long term nature of seal when place under a thin facial muscle. Absorbable cyanoacrylates such as OMNEX may be preferred based on reversibility and lack of long term potential for scarring and foreign body reactions. The use of cyanoacrylate is described in more detail below.

### Other Types of Bioadhesives

In other embodiments, plasma polymerized N-isopropyl acrylamide (pNIPAM) or ONYX™ are suitable bioadhesives for the present invention.

### Use of Cyanoarcrylate Derived Glues as an Injectable Agent for the Purpose of Altering Myo-Osseous Fixation

Cyanoacrylate glues are well known adhesives which are known to produce strong bonding during polymerization and forting tight bonding with a degree of heat release. Medical applications have commonly included skin approximation as an alternative to conventional sutures for which the material is currently commercialized. Deep tissue implantation has also been advocated for neural trunk repair during soft tissue surgery, vascular repair, fixation of extraocular muscles to the ocular globe or implanted prosthesis after globe removal, embolization for vascular malformation using neuroradiologic techniques.

Major concerns from regulators have included excessive inflammatory reactions from chemical breakdown of implanted glue which can form formaldehyde, a chemical toxic to tissues. Heat generation during polymerization and hardening has also been noted but represents a lesser problem. Efforts have been made to find cyanoacryolate derivative which have a reduced rate of formaldyhyde generation. Non-limiting examples of cyanoacrylastes suitable for use with the present invention included: alkyl 2-cyanoacrylate, alkenyl 2-cyanoacrylate, alkoxyalkyl 2-cyanoacrylate, and carbalkoxyalkyl 2-cyanoacrylate. The alkyl group may have 1 to 16 carbon atoms and is preferably a C₁-C₈ alkyl 2-cyanoacrylate. Suitable cyanoacrylates include, for example, methyl 2-cyanoacrylate, ethyl 2-cyanoacrylate, n-propyl 2-cyanoacrylate, iso-propyl 2-cyanoacrylate, n-butyl 2-cyanoacrylate, iso-butyl 2-cyanoacrylate, hexyl 2-cyanoacrylate, n-octyl 2-cyanoacrylate, 2-octyl 2-cyanoacrylate, 2-methoxyethyl 2-cyanoacrylate, 2-ethoxyethyl 2-cyanoacrylate and 2-propoxyethyl 2-cyanoacrylate, methyl.alpha.-cyanoacrylate, ethyl.alpha.-cyanoacrylate, propyl.alpha.-cyanoacrylate, butyl.alpha.-cyanoacrylate, and cyclohexyl.alpha.-cyanoacrylate, alkenyl and cycloalkenyl.alpha.-cyanoacrylates such as allyl.alpha.-cyanoacrylate, methallyl.alpha.-cyanoacrylate, and cyclohexenyl.alpha.-cyanoacrylate, alkynyl.alpha.-cyanoacrylates such as propargyl.alpha.-cyanoacrylate, aryl.alpha.-cyanoacrylates such as phenyl.alpha.-cyanoacrylate and toluoyl.alpha.-cyanoacrylate, hetero atom-containing methox yethyl.alpha.-cyanoacrylate, ethoxyethyl.alpha.-cyanoacrylate, furfuryl.alpha.-cyanoacrylate, silicon atom-containing trimethylsilylmethyl.alpha.-cyanoacrylate, trimethylsilylethyl.alpha.-cyanoacrylate, trimethylsilylpropyl.alpha.-cyanoacrylate and dimethylvinylsilylmethyl.alpha.-cyanoacrylate.

A preferred cyanoacrylate is 2-octyl)-cyanoacrylate because of its slow degradation and reduced rate of formaldyhyde generation.

The surgical method of claim 1, wherein said bioadhesive has a slow degradation and reduced rate of formaldyhyde regeneration.

It is anticipated that multiple forms of delivery devices may be used to inject adhesives through a puncture site for the purpose of glue placement in the defined fibro fatty plane on the undersurface of the muscle tissue. These include prefilled syringes with sterilized liquid adhesive which remain air tight until the moment preceding use. Tube enveloping containers capable of delivering a fixed volume of liquid adhesive also may be used. Diaphragm shaped containers containing a fixed amount of glue may also be used. Any container forms mentioned are not limiting as multiple devices are possible so long as the device maintains sterility of the injectable and is able to deliver a fixed and consistent volume of adhesive agent.

### Absorbable Type Cyanoacrylates

A class of cyanoacrylate glues characterized as “absorbable tissue glue” has been advanced and advocated. A limiting factor of cycanoacrylate glues is granuloma formation from breakdown into toxic subcomponents such as formaldehyde. Glue designed to absorb with lesser chances of foreign body inflammatory reaction have lesser chance to induce this form of complication and have improved tolerability. Although the mechanical adhesion may be temporary, adjuctive inflammatory reaction from single or multiple injections can induce fibrosis which can function as an endogenous glue and adhesive achieving the same effect as an foreign substance. The net effect would be to create a more permanent or lasting adhesion. The tolerability of absorbable cyanocrylates will result in less secondary inflammatory complications, less scarring and an improved side effect profile than some of the conventional preparations listed above. Currently, Omnex™ from Johnson and Johnson represents an example of the absorbable cyanoacrylate class appropriate for the practice described herein. Absorbable class glues are not to be limited by one formulation or brand as other forms of absorbable bioadhesives can be used to achieve similar results.

### Alternatives to Cyanacrylate Derived Glues

Because of the high degree of tissue reactivity induced by some implanted cyanoacrylates, alternative forms of liquid or gel bioadhesives can be applied for the same purpose and delivered by syringe and puncture site. Alternative materials may include various forms of silicone, acrylic or any other substance demonstrated to have less than 5% induced inflammatory rate. Devices may be injected through puncture sites to achieve the same end. These devices must be implable through a microincision or puncture, be capable of fixation the undersurface of muscle to bone by elevation and fixation of the undersurface of the muscle to bone and tether muscle to bone. The device is placed through the skin and inserted between the soft tissue and the bone. In one embodiment, a device is a linear implant with extending side barbs. The extending side barbs may be hard and stiff, and may be of various lengths relative to the length of the linear implant.

Method of Tissue Manipulation after Injection of the Liquid Adhesive

Once the clinician injects the liquid adhesive, it would be important to manipulate the soft tissue overlying the bony surface over which the injection has been administered so that the alteration in contour and suspension is placed in a desirable configuration. For example, the method of application using a liquid adhesive would be organized in the following steps:

(1) Small amount of anesthestic (eg lidociane) injected over the desired bony surface within the low resistance fibro fatty tissue planel; (2) Injection of the liquid adhesive agent (eg Cyanoacrytlate glue) over the bone in the fibrofatty plane on the undersurface of the muscle, through a puncture site, not involving an incision in one embodiment, or involving a microincision in another embodiment, so that harding and polymerization is initiated. Care is taken to avoid direct injection into nerve, arteries veins, or other anatomically sensitive areas so as to avoid complications; (3) Tissue manipulation by the clinician externally so that the soft tissue can be repositioned or resuspended into a desirable configuration. For instance, frontal bone injection can be followed by manual elevation of the brow to achieve a brow lift. Maxillary and zygomatic bone injections can be administered so as to achieve midface lift by manually elevating the check from the surface over the maxillae and zygomatic bones. Mandibular inferior border, anterior or posterior surface can be injected followed by manipulation of the soft tissue of the jowl region so as to resuspend and tighten the jowl region so as to reshape the jowl to a more contoured and youthful configuration without the use of conventional incisional plastic surgery of the face; and (4) Other injection location in non facial areas of the bony and the concept and technique should not necessarily be limited by exact anatomic relationships.

### Amount of Injectable Glues and Adhesives Used

The amount of glue or adhesives used is limited by the critical amount which will cause inflammatory, granulomatous reactions or excessively cause tissue contractions with dimpling of the skin surface. In one embodiment, the amount may vary from between about 0.001 mL to about 10 mL per injection site. In other embodiments, the amounts may vary (all given on a per injection site basis) from between about 0.001 ml to about 8 mL, from between about 0.001 mL to about 6 mL, from between about 0.001 mL to about 4 mL, from between about 0.001 mL to about 2 mL, from between about 0.001 mL to about 1 mL, and from between about 0.001 mL to about 0.5 mL. In a preferred embodiment, the amount per injection site is between about 0.1 mL to about 0.2 mL. Multiple injections are needed depending on the targeted region and strength of adhesion sought per given anatomic region. Multiple injection point per region are anticipated.

### Device Used for Injections

In one embodiment, the device comprises a single use device comprising a needle or micropuncture device, with a bioadhesive, for example but not limited to, cyanoacrylate glue. Other forms of bioadhesives or tissue glues such as thrombin fibrinogen may involve compounded needle bores with individual ports corresponding to a given component of the glue substance needed for curing. A syringe with predetermined injection needle or micropuncture would be the preferred embodiment of the delivery system with the intention for single puncture per syringe use. Alternatively a multiple use delivery syringe may be used for more rapid administration techniques.

### Intramuscular Injection of Glue to Establish Muscle Fiber Support Compaction and Connective Tissue Reinforcement

In addition to muscle fixation to bone, some embodiments of the present invention may make use of intramuscular glue to establish a support to the endomysium, perimysium, and/or other intramuscular and intermuscular support of connective tissue. The function is to compact muscule groups, alter intermuscular adhesions, and/or elevate muscular dissent with respect to bone and intermuscular attachments for the purpose of elevating, tightening and altering the contour of surface structures for aesthetic purpose. An example of this type of application is gluing the submental platysmal muscle to the digastrics muscles for the purpose of improving the angulation between the neck and undersurface of the jaw. The glue in this situation is placed between a flat flimsy muscles which is poorly compacted by aging collagen within the muscle proper to the well compacted and well supported deeper muscles effectively compacting the muscle groups and achieving a more youthful surface appearance. Similar enhancement of the jowl tissues to deeper muscle fascia of the temoralis muscle can be used to elevate the jowl region and gluing the lower portion of the jowl to digastric, sternomastoid, sclene muscles effectively achieve the same purpose. Glue may be placed over the masseter muscles, into adjacent parotid gland, or related fascial surfaces.

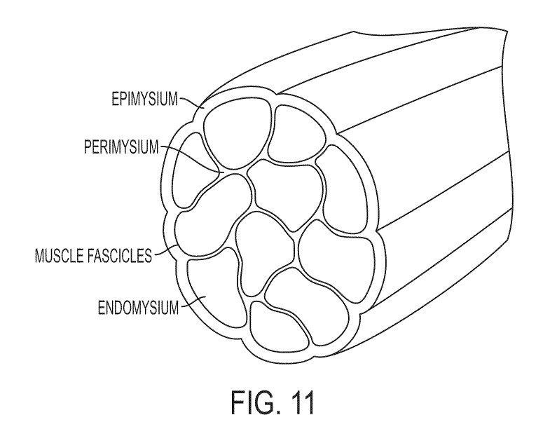

Direct injection of glue into muscle functions to causes a compacting effect on the muscles volume. This is accomplished by providing a sunthetic matrix which function like the natural endomysium of the muscle as shown in FIG. 11. The muscle endomycium is structurally supported by collagen protein which loosens with age. The synthetic matrix serves to reinforce the lossening supporting structure within the muscle allowing the injecting physician and surgeon to control the muscle volume and supporting surface contour formed by muscle volume. The glue needs to be diffusely distributed throughout the target muscle followed by external surface manipulation to shape and contour the muscle to an appropriate and desired configuration. Care must be placed not to allow glue to cure at puncture or micro incision sites as healing can be impaired allowing a conduit for infection.

Bioadhesives injected via puncture with needle or micro incision within muscle can function as a synthetic perimysium, endomysium and epimysium for a targeted muscle or muscle group. Bioadhesive injection directly into a muscle can create a restriction of contractility, hence this method may be useful in the therapy for disorders involving involuntary movement such as spasticity, dystonia, painful reflex muscle spasms, myofascial pain, bruxism, temporal madibular joint syndrome, twitches and other facial spasms, posture deformity, cerebral palsy, spasmodic disorders of growth and development such as scoliosis and other spasm types.

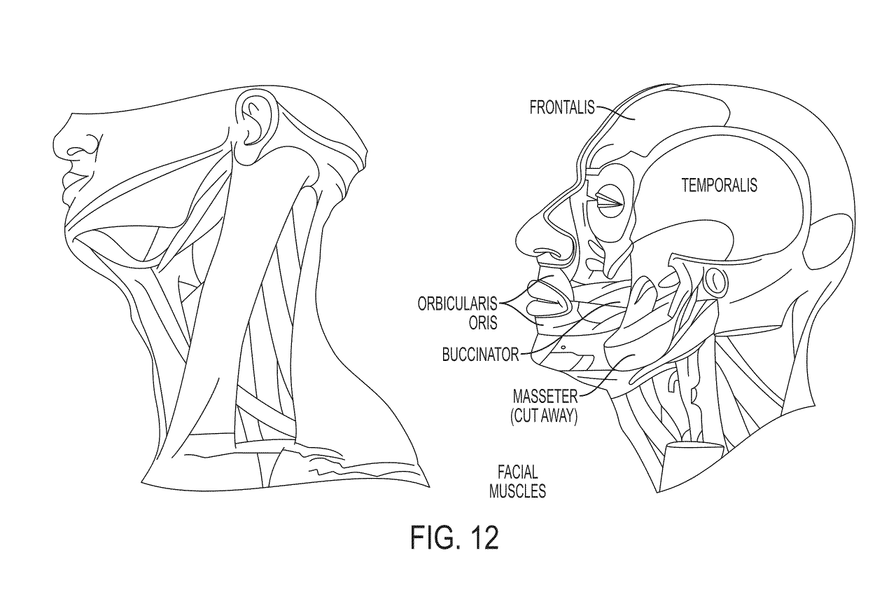

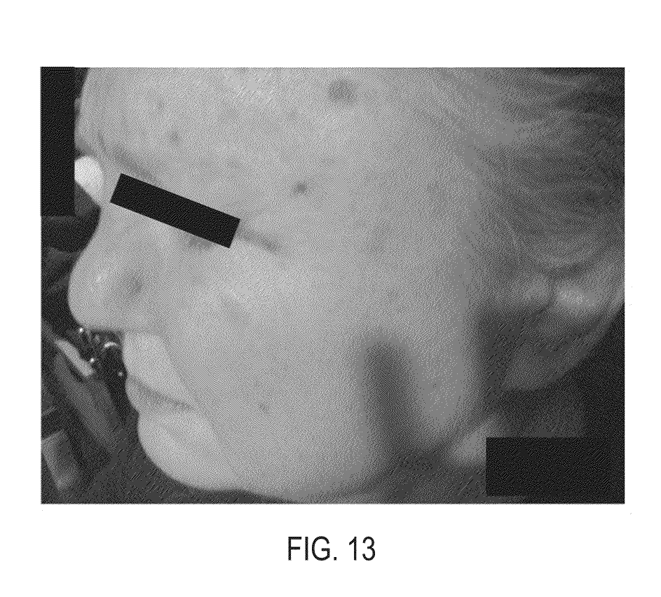

With reference to FIG. 12, arrows point to areas where glue can influence intermuscular adhesion and intramuscular cohesiveness to effect contour and surface changes on the human head and neck region. The glue may be for instance, placed under the platysma muscle to flatten neck bands, to decrease volume of the platysma muscle, and to effect a compression of the platysma muscle to the firmly adherent digastrics muscles and muscles in proximity to the hyoid bone, causing a more youthful acute neck angle and a reduction in the hanging jowl. As another example, deeper injection of glue into the masseter muscle can be used to compress this muscle reduce its volume and reduce a square lower face deformity commonly seen with aging. The masseter muscle accounts for a substantial portion of the lower face volume.

Other anatomic regions may be approached in similar manor and concept and application should not be limited by region.

### EXAMPLES

### Example 1

### Mid Face Lift

The methods and materials described herein are ideal for accomplishing a mid face lift. The SMAS layer encompasses the zygomatic major and minor muscles as well as adjacent facial muscles. The malar eminence is formed by the zygomatic bone which contains no essential arteries or other neurovascular structures hence this bony platform is ideal for fixating the muscular structures. The zygomatic major and minor muscles are impaled with associated SMAS with the self-tapping or self drilling or self drilling screw via a small incision site over the lateral orbital region in the vicinity on or superior to the zygomatic bone. The muscles associated with its surrounding fascia are elevated and fixated superior to the malar eminence effectively lifting the attachments (insertions) of the zygomaticus muscles (malar eminence) and fixating the soft tissue lift to the facial bone. Once fixation has been established supporting fixation can be accomplished by use of one or more additional self tapping or self drilling compression fixation bolts to the bony platforms in this region. Additional self tapping or self drilling bolts may be placed in small incisions in the nasolabial fold, or inferior orbital rim, along natural cutaneous tension lines in order to increase fixation under small cutaneous puncture sites.

### Example 2

### Adaptability of Techniques to Other Body Regions—Breast Ptosis

The concept of osseous soft tissue integration under a screw head or compression plate can be applied outside the facial regions. For instance, age-related breast sagging (breast ptosis in the female) is considered a culturally undesirable senescent change. Use of compression fixation to the clavicle via deep fascial can serve as a method to treat breast ptosis. Ptosis, defined as progressive dropping of the apex of the breast with age, can be modified with volume expanders such as breast implants (for example, silicone gel implants) as well as superior suspension with fascial fixation. Additionally, osseous fixation can be used to modify breast contour, that is relative configuration of tissue curves by fixation to ribs, sternum or clavicle. Small incisions can be made under the clavicular region and the superficial and/or deep fascial can be impaled by the self-tapping or self drilling screw system and fixated to the inferior border of the clavicle creating effectively a breast lift. Self-tapping or self drilling screws within this anatomic region for such a procedure by necessity should be longer than those used on the facial region so that this larger anatomic region can be adequately fixated. Fixation point should be the inferior, inferior anterior or anterior surface of the clavicle, the rib or the sternum. The clavicle and sternum are relatively immobile fixation points (as are the facial bones and pelvis) and serve as excellent regions for soft tissue osseous fixation under a compression plate or screw head.

### Example 3

### Adaptability of Techniques to Other Body Regions—Buttock Elevation

Buttock elevation and contour changing can be accomplished with osseous fixation using compression plates or self-tapping or self drilling screw fixation to pelvic osseous ridge both superiorly and posteriorly. Use of soft tissue compression over regions not containing critical nerves or vessels allows safe fixation tissue contouring and/or contour curve alterations. Adjunctive elements to the compression plates may include suspension cables passed through soft tissue to achieve a greater lift. The cable extensions may be fixated to the compression screws.

### Example 4

### Adaptability of Techniques to other Body Regions—Supra Pelvic Lateral Bulge (Love Handles)

Alteration of bulge around the waist line may be accomplished via multiple soft tissue compression plates to the upper portions of the pelvis effectively reducing waist bulge. The same concept of fixating soft tissue via small incisions into the bone via a self-tapping or self drilling screw system is used to re-contour and re-suspend soft tissues into the pelvic rim altering the protrusion of the waist line, an age-related anatomic finding. The compression screws may be used in addition to existent methods of liposuction and other forms of adipose resection, thermal reductions, ultrasonic or radiofrequency fat reduction.

### Example 5

### Adaptability of Techniques to Other Body Regions—Floppy Posterior Arm Soft Tissue Contour Fixation

Floppy posterior arm syndrome is a cosmetic deformity noted by many middle aged women. Efforts to treat this condition with radiofrequency have often failed. The use of a soft tissue fixation-compression plate or screw bolting arrangement may be used to alter the contour and increase support in this region to reduce the deformity. Care must be taken not to impale critical nerves or vessels into the extremity.

### Example 6

### Use of Suspension Cables Fixated to a Soft Tissue Oseous Fixation Plate

Soft tissue fixation may be enhanced using a tissue fixation cable passed through a soft tissue structure and stabilized under a fixation plate. An example would be a non absorbable material such as orolene fixated to a compression plate, or screw head on the underside of the mandibular bone to reduce jowl contour with the fixation cable passed across the submandibular region to the fixation screwhead on opposite side to sling the submandibular region causing enhancement on angulations of the neck, an aesthetically more youthful appearance. Here the fixation screw not only improves the contour of the jowl region but also serves to be an anchor for a suspension cable or suspension mesh.

### Example 7

### Facial Palsy Applications

Chronic facial palsy can have major impact on quality of life resulting from disfigurement from asymmetric facial movements and laxity of facial structures at rest. The latter situation results in brow ptosis, dropping angle of the mouth and cheek asymmetry. Reasons for chronic facial palsy include Bell's palsy, traumatic skull injury, surgery (for example, acoustic neuroma resection), Ramsy Hunt syndrome, Mastoiditis. Lyme disease, stroke as well as other nosologic entities.

### Example 8

### Treatment of Brow Ptosis, Cheek Ptosis and Asymmetry of the Lower Face

The compression plate and screws are placed in a similar manner as described above to achieve an enhanced symmetry at rest for such patients. Preferred areas to accomplish connective tissue fixation include frontal bone for brow ptosis, lateral orbital rim for cheek ptosis, maxillary face (anterior maxillary bone for angle of the mouth). Positioning of the fixation points needs to be individualized for each patient after an assessment of the regional severity of the deficits.

Treatment of paralyzed lid closure associated with facial paralysis often involves the use of an implantable gold weight into the upper lid which serves to aid in closure of the eye with the normal blink. The weight works on the principle that the levator palebrae superioris muscles have the natural brainstem impulse momentarily turned off during lid closure occurring during a natural blink. The gold loaded lid is much heavier than the normal lid and the weight is projected away from the fulcrum point on the superior surface of the eye, hence torque is established causing lid closure when levator tone is interrupted. A closing eyelid during blinking is critical as the continuity of the tear film needs to be physically maintained over the cornea for transpiration of oxygen and to protect the epithial integrity of the optical surface. Loss of an ultrasmooth optical surface results is optical ray diffraction with irregular astigmatism, surface ulceration, scarring, neovascularization and potentially permanent visual loss.

The compression screw procedure represents a method to tighten the lateral reticulum (and lateral canthal tendon) which serve to further enhance the effect of implantable gold weight on lid closure by tightening the horizontal support of the upper and lower eyelids. Furthermore, in this situation, the lower lids can be elevated, further enhancing corneal protection for the paralyzed face. In summary, the compression plate and screw heads can be used to tighten and re-suspend the lateral orbital retinaculum and lateral canthal tendon as well as the sub-orbicularis fascia effectively creating more protection of the ocular surface from exposures due to paralyzed eyelids. The procedure can be updated over time to maintain the benefit of the correction.

### Example 9

### Blepharospasm

Benign essential blepharospasm is a condition characterized by involuntary closure of the eyelids in the absence of primary eye disease. This condition, although treated with various medications, often requires surgical management. The surgical management may include brow lift. The condition is also characterized by a phenomenon known as geste antagonist. Geste antagonist is a French derivative word construction to describe a rubbing gesture on the brow or near the brow region which mitigates the involuntary movements. The compression screw or compression plate can provide a nidus or point source for sensory stimulation often self-applied by the patient to relieve symptoms. Additionally brow lifting surgery is often a component of various surgical procedures which are used for therapy of this condition. The compression plate has been used in complicated management problems in patients afflicted with blepharospasm who have failed conventional therapy. The procedure is minimally invasive with quick recovery time.

### Example 10

### Use of Myo-Osseous Fixation to Remove Facial Wrinkles

Myo osseous fixation accomplished by screw fixation devices, various forms of tissue glue, or scarring sclerosis agents or other methods to fixate facial muscles to bone can be used as a method to reduce static and dynamic frown lines crows-feet lines, transverse forehead wrinkles, cheek wrinkles, as well as alterations in facial cheek, jowl, chin, and lip contours.

The principle creating this effect is weakening muscles that create lines in the surface associated with facial expression. Such lines and wrinkles are associated with aging and suppression of the appearance of such lines has been deemed pleasing to many of the middle aged and elderly population. Dynamic lines are associated with the contraction of muscle, for example during smiling, crows-feet lines may appear. Static lines are those that are not associated with contraction of muscle.

Fixation of muscles into underlying facial bones using implantable screws, or other agents which fixate facial muscles to bone or obliterate the freely moveable pre-periosteal fat pads of the facial bones, causes a relative reduction in facial tone and possible resting facial tone therefore, reducing the dynamic rhytide (facial wrinkle) associated with facial muscle actions. The mechanisms of the facial muscle mini-bolt or other forms of alteration of facial muscle fixation causes a longer action, more permanent effect on the fibro fatty tissues underlying the facial muscles, thereby creating a more permanent mechanism for reduction of facial muscle contractility, and a more lasting reduction of facial rhytides.

Not to limit the application of this concept by example, crowsfeet can be eliminated or reduced by pinning lateral orbicularis muscle to lateral orbital rim or surrounding bones, glabellar lines can be limited by fixating glabellar muscles to the glabellar bone, and frontalis fixation into frontal bone can limit transverse forehead creases.

### Example 11

### Use of Tissue Glues to Fixate Facial Muscles

Other than tissue fixation screws or bolts, fixation glue—like substances can be used to fixate facial muscle to bone, as well as create scarring in the fibro fatty lubricated sub-muscular tissue plane which can result in decreased facial motility, reduce unwanted facial movement and treat static and dynamic facial lines (such as those in the facial glabellar region, lateral orbital crow's feet, and the transverse lines in the forehead).

The principle creating facial lines is weakening muscles that create lines in the surface associated with facial expression. Such lines and wrinkles are associated with aging and suppression of the appearance of such lines has been deemed pleasing to many of the middle aged and elderly population. Dynamic lines are associated with the contraction of muscle, for example during smiling, crows-feet lines may appear. Static lines are those that are not associated with contraction of muscle.

Tissue glue can be made of various substances, such as polymethyl methyacrylate (PMMA) (“superglue”), cyanoacrylates as well as a fibrinogen fibrin conversion with human thrombin. Such glues may be used in addition to bolting devices to achieve a reduction in facial tone, movement, and surface wrinkles as well as to alter facial configuration such as contour, cheek browlift, chin alteration, lip augmentation. Currently tissue glue is available as Tisseel™, (fibrinogen-thrombin to fibrin) double chamber arrangement, 2 octly cyanoacrylate, n-butyl cyanoacrylate, as well as other formulations of cyanoacrylate mentioned herein.

The use of glues and adhesive devices within the pre-periosteal fat layers of the facial bone can be useful in altering facial contours such as configuration and shape of the malar eminences, jowl configuration, forehead configuration as well as submental contours (under the mandible and chin). To accomplish alteration in jowl configuration and hanging fixation is accomplished using between 1-8 compression bolts on each side (preferably 1 or 2 per side) fixated to undersurface, outer surface or internal surface of the mandible, with care taken to avoid the mandibular branch of the facial nerve. The location best be close to the symphesis of the mandible or the angle of the mandible. Alternatively bioadhesives may be injected followed by mechanical support allowing fixation of the facial muscles to the mandible and therefore altering configuration of the jowl. Mechanical support may involve a temporary compression bandage or manual fixation and support by the surgeons hand. Other than jowl, submental skin hang “loose turkey neck syndrome”, can also be treated with submental support allowing facial muscle adhesion to bone. Wrinkles within the neck region may also be addressed with a similar method. Lip rotation may also be accomplished allowing for eversion of the red fleshy lip by fixing the orbicularis on muscle to bone. It is anticipated that various method of face immobilization with bandages or temporary support devices or use with other methods may be necessary to sustain the desired effect while the bioadhesive cures, and an appropriate level of fibrosis around the injection site ensues.

It is apparent that glues may be used independently or with other fixation devices such a bolts, screws, screw washer arrangements to achieve, screw with expendable heads, or fixation plates. Glues or other fibrosis inducing agents can be place under the facial muscles producing the dynamic lines so that the lines or wrinkles can be reduced.

### Example 12

### Myo-Osseous Integration with Compression Plate for the Treatment of Blepharospasm (Specific Procedure)

### Pertinent Surgical Anatomy Relative to the Procedure

The orbiclaris oculii is conceptually divided into three components, the pre-tarsal, pre-septal and the pre-orbital components. The pre-tarsal and pre-septal components generally contribute to voluntary and involuntary blinking whereas the pre-orbital contributes to voluntary squeezing of the eyelids. Involuntary contraction of the pre-orbital components accounts for the severe squeezing associated with various forms of involuntary blepharospasm and serves as the target for the surgical modification.

The orbiclaris muscle is a sphincter muscle receiving innervations from temporal, zygomatic and buccal branches of the facial nerve and originates from the maxillary process of the frontal bone and the medial canthal tendon, fibrous lacrimal sac, and posterior lacrimal crest. The pre-tarsal and pre-septal areas intertwine at the lateral raphe whereas the pre-orbital component sweeps around the lateral orbital region forming a continuous sphincter.

Contraction of the pre-tarsal area creates force and increased anterior posterior torque at the tarsus relative to the globe causing uniform eyelid closure. Contraction of the orbicularis causes a concentric movement of the brow, temporal soft tissue and cheek toward the medial canthal region causing a tight palpebral closure. The concentric closure of the pre-orbital section is dependent on the loose attachments to underlying bone and loose network integration with the superficial muscular aponeurotic fascial system (SMAS).

The orbicularis muscle superiorly and super temporally is tightly attached to the dermis on its superficial surface and is separated from the bone by a fat pad and the galea aponeurotica. The galea aponeurotica and the eyebrow fat pad above the orbital rim are loose allowing for a “lubricated slide” during frontalis contraction to achieve brow elevation during facial expression. The galea aponeurotica divides into superficial and deep layers which engulf the orbicularis and frontalis muscles above the brow in the forehead region. The posterior galea is attached more firmly to the inner two thirds of the orbital rim with almost no attachment to the lateral orbital rim.

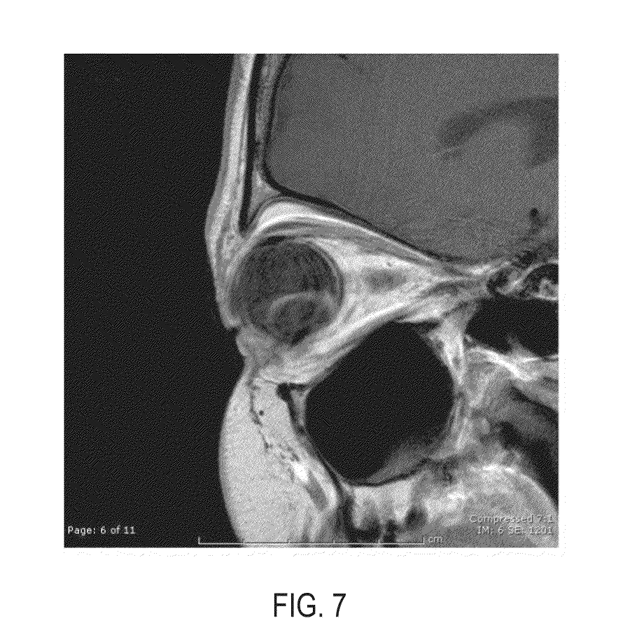

The functional consequence of this anatomic configuration is to create a muscular system with dermis and skin which roll over a loosely “lubricated” fibro-fatty surface allowing for the ease of brow excursion (FIG. 7). The ease of excursion is greater lateral than medically as the aponeurotica is more firmly attached medially offering a check to muscle driven elevation (and decreased decent with aging). The configuration and friction reducing effect of the sub galeal aponeurotic fat is essential to the free movement of the forehead by the frontalis muscle and brow contractions by the orbicularis muscle.

Pre periosteal fat in other regions of the face allows for variable points of reduced resistance allowing for active movement during various facial expressions. For instance, cheek elevation during active zygomaticus muscle group contraction is facilitated by the loose pre-periosteal fat pads of the maxillary face. For the orbicularis and frontalis facial muscles, the fat pads allowing for low friction movement along the upper orbital rim are identified with arrows on a T1 weighted surface coil MRI noted below (FIG. 7). Fat interposed between the anterior surface of the frontalis and orbicularis muscle further can contribute to reduction in muscle resistance and also facilitate the effect of facial muscle contraction on facial expression, brow elevation, blink function, and other forms of facial movement. A graphic of the extent of the pre-periosteal, sub-aponeurotic and sub dermal fat can be seen on the magnetic resonance image of the forehead scalp and skull table. The T1 weighted image demonstrates the high contrast fat layer in the pre-periosteal, sub-aponeurotic region and in the anterior region of the orbicularis frontalis muscle region above the eyebrow.

Myo-osseous fixation for the treatment of hanging jowl is accomplished by fixation to the border of the mandible close the its symphesis, or angle with efforts to avoid damage to the mandibular branch of the facial nerve or other sensory nerves in the region. The injection will involve the mandibular border, anterior or posterior surface. Multiple injections points are usually necessary and multiple treatment cycle during various time intervals would be needed in many cases. The improvement in method over conventional facelifts related to the elimination of the surgical incision, use of extensive suturing, and extended post operative heal time as well as the ease of multiple touch up or maintenance injections session. The effect of reducing the procedure to a needle puncture and injection of a glue materials or small implantable screw allows for a very short post operative period with reduced pain, edema, erythema, and disfigurement associated with conventional surgical facelifts. Even in the situation of inconsistent muscle bone adherence, the opportunity to easily repeat the procedure without the morbidity of incisional surgery is ever present.

### Basic Concept of the Procedure

The functional concept of the orbicularis fixation procedure is may be described as the following:

(1) To dampen and alter the force vector created by contraction of the muscle by modifying its attachments to the bone and creating adhesions and resistance to the muscle's undersurface via scar; and (2) achieve a brow lift.

The orbicularis muscle is fixated to skin on its superficial surface and more importantly overlies a fibro-fatty layer on its undersurface consisting of posterior extensions of the galea aponeurotica and brow fat pads. The layers of the brow can be grouped into a tightly connected skin, orbicularis-frontalis units overlaying a loose fat pad galea unit. The motility of the brow is dependent on both muscle contraction as well as a low resistance undersurface of fat and galea. Brow and lid movement contraction of the orbicularis is dependent on the ease of slide between the skin-orbicularis-frontalis unit and the underlying fat pad-galea unit.

Compressing and fixating the orbicularis muscle directly to bone alters this low tension plane causing resistance to low tension slide and movement between these functional tissue planes. Increasing frictional resistance via fixation and scaring serves to decrease the force generated by the muscle.

Direct immobilization of the orbicularis muscle shortening via fixation of its segments by pinning or gluing (for example with bioadhesive) the pre-orbital portions to the frontal bone represents another mechanism by which the procedure dampens the contractility of this muscle. Elevation of the superior portion of the muscle also may serve to alter fiber angulations contrary to optimal force generation. The horizontal fibers directed perpendicular to the eyelid fulcrum are now directed diagonally causing an alteration in force component directed toward the vertical plane.

Although it is not clear at this time which mechanism is most important, the net effect is to reduce forced closure of the upper eyelid during involuntary contractions facial contractions without impairing the natural blink movements associated with the eyelid portions of this muscle.

Beyond muscular effects, the sensory and proprioceptive effects of orbicuclaris fixation may play a significant mechanistic role. Fixation of a muscular segment will modulate proprioceptive output as well as create alteration in sensory nerve interplay, similar to the auto stimulation self induced by many patients with essential blepharospasm (geste antagonist).

### Description of the Procedure

Three horizontal 2-3 mm marks are made equidistant along the superior cilia of the brow at the medial position, mid position and lateral position. Doppler flow meter is used to identify the superior neurovascular bundle to avoid positioning the compression screws directly over the superior ophthalmic artery. The brow area is infiltrated with a 50:50 solution of 2% lidocaine and marcaine containing hyaluronidase at a concentration of 5.0 U/cc. About 3-5 cc's of anesthetic per brow are injected along the superior orbital rim on each side and about 10 minutes with gentle pressure is allowed to elapse. Incisions are made through epidermis and superficial dermis, taking care not to penetrate the dermis and impale the orbicularis muscle. The orbicularis muscle is densely attached to dermis in this region and connective tissue under the head of the screw facilitates the muscle fixation.

Synthes™ miniplate unit containing 1.0-1.3 mm by 3 to 4 mm length self tapping or self drilling titanium screws with 1.0-1.3 compression wrench are the vital instruments necessary for this procedure. The compression wrench engages the screw head firmly and functions much like a needle holder for this procedure. The brow is maximally elevated toward the vertex of the skull as the 1.0 or 1.3 mm screw loaded in the compression wrench impales the deep dermis, orbicularis, galea and sub galea fat driving the self tapping or self drilling screw directly into the outer table of the frontal bone.

The Philips head driver wrench is rotated clockwise boring a track into the frontal bone between 2-3 mm in depth, producing a tight fixation between the orbicularis and bone. The screw head is rotated firmly to insure a flush relationship with the bony plane (failure to do so results in a palpable even visible bump, a technical complication). Skin distortion around the entry point is eliminated by gentle undermining of the surrounding dermis. Care is taken not to produce excessive pressure on the wrench as the out table of the skull can infracture producing more post operative pain. Screws with a length greater than 5 mm may penetrate through the diploe into the posterior table of the frontal bone causing increased post operative pain and increases the risk of full thickness skull table penetration.

The brow is generally more difficult to elevate medially because of tighter attachments of the galea aponeurotica to the medial orbital rim, however, elevation is still possible. Over-correction is desirable and descent of the brow tends to occur over a two-week period post-operatively. Generally three screws per brow are used for the male brow and two screws are used for smaller female brows. A fourth screw may be considered along the lateral orbital rim for severe cases. Skin closure is accomplished with 1, 6-0 silk suture per puncture site.

### Example 13

### Myo Osseous Fixation for the Purpose of Altering Facial Contour and Wrinkles without the Use of a Fixation Device

As cases have been observed in which the muscle to bone fixation with a titanium screw and plate needed to be removed after a period of several months, the opportunity presented to observe whether the residual scar can hold the muscular tissues and SMAS to the bone. In these cases, it was noted that tissue elevation could still be achieved and that a scar over the fixation device could be imaged on computerized axial tomograms. This observation indicated that (1) a permanent fixation device may not be necessary and that (2) fixation may be able to be achieved with a fibrosis agent or plate without direct drilling into bone. The fibrosis agent may include injected glues, or fibrous provoking materials placed for the limited purpose of causing an adhesion of the undersurface of facial muscle to facial bone beyond the usual extent. Such a closed system procedure can decrease contractility of the muscle by causing an undersurface adhesion, and therefore alter the dynamic lines seen with aging of the human face.

### Concept of Therapeutic Methods for Headaches Using Implanted Devices

Therapeutic methods and devices for subjects afflicted with a human headache disorder, such as migraine or other headaches, are disclosed. The methods involves stimulation of the trigeminal nerve for the purpose of aborting the headache and treating the associated pain. The methods and devices allow for a safe, minimally invasive, non pharmaceutical-based therapy to abort and mitigate migraine pain. These devices and methods allow for a convenient form of therapy without systemic side effects.

### Variants of Migraine Headache

Migraine headache is generally defined as an episodic, severe headache, lasts 4-72 hours, and is associate with light and sound sensitivity and varying degrees of nausea and occasional vomiting. The pain is often unilateral but can be bilateral and is often worsened by movement. The variation of migraine can occur with respect to neurologic deficits seen early in the headache attack, association with other forms of headache (eg. tension headache, myofascial headache).

The variants of migraine can be often defined by vasospasm associated with the early phases of the headache, which can create transient neurologic deficits such as loss of vision or visual field, visual hallucinations, impaired speech (dysarthria, aphasia), paralysis, sensory loss, loss of consciousness, and nerve palsy (eg. oculomotor nerve palsy).

Other variations of migraine can be associated with frequent attacks which are thought to cause a chronic sensitization of pain center in thalamus and related brain structures causing chronic daily pain, characterized as chronic daily headaches, myofascial pain, and fibromyalgia, associated with the more severe migraine syndrome.

Other variations include extended repetitive headaches over a period of weeks (cluster headaches), facial headaches (sinus migraine), migraine associated with acute allergy (hay fever), migraine associated with menstruation, migraine associated with Horner's syndrome, retinal migraine, and food associated migraine syndrome (eg associated with consumption of wine and cheeses, monosodium glutamate, bisulfate).

The methods disclosed herein are intended for use in many variants of migraine. A preferred embodiment of the method is a treatment for the common migraine as defined in the international headache society classification under section 1. See Table 1. Another preferred embodiment of the method is a treatment for tension type headaches as defined in the international headache society classification under section 2.

### Sensory Tactile Suppression of Headaches

It has been long known that some human essential headache disorders, which include migraine, tension headache, or a combination of both, as well as cluster headaches and other headaches not associated with structural lesions of the brain that are listed in Table 1 are associated with the presence of tactile suppression. Tactile suppression involves stimulation of the forehead, usually self stimulated with digits or pressure, which relieves the pain to some degree. The common migraine patient will often press on their head or put objects on their head, such as a cold cloth, to press on their head to sustain a relief in pain. This phenomenon has also been seen with movement diseases of the face, head, and neck, such as the sensory suppression and sensory tricks associated with adult onset spasmodic torticollis and facial dystonia and blepharospasm in Meige syndrome. Self stimulation has been thought and described to suppress interneuronal activity of brain stem neurons associated with movement in these situations (see Tolosa et al). The suppression seen with human headache disorders has not been extensively studied from a neurophysiologic point of view, but likely represents a similar type of response as seen with human movement disorders. This would be interpreted as a sensory stimulation of the head and neck region and headache disorders, or suppresses upregulated hyperactive neurons in the brain which generate the pain. These neurons probably occur in the thalamus, but has often been thought to spread to multiple areas and does not represent one specify loci. The tactile stimulation is simulated in an extreme form by the use of acupuncture.

Acupuncture has been shown to be effective in the treatment of severe pain and headaches in a clinical setting. Acupuncture in a patient with a severe headache can be relieving of the pain and mitigate the degree of symptoms. The American Academy of ophthalmology in 2003 has supported the use of acupuncture for the treatment of headache disorders based on clinical experience and some degree of studies. The disclosure of this document is incorporated herein in its entirety. See Complementary Therapy Assessment Acupuncture for Ocular Conditions and Headaches, American Academy of Opthamology, May 2003, the disclosure of which is herein incorporated by reference.

The methods disclosed herein build on the principle that acupuncture can be found to be effective in both abortive therapy and pain therapy. In abortive therapy, during the prodromal period of the migraine, acupuncture can be applied to abort the full development and evolution of the pain syndrome. Generally the needles are placed in the temporal regions of the face, hairline of the facial, temporal, and neck regions. The needles are generally placed from 1 to 100 positions, but preferably 2 to 6 needles are used in the punctures. The purpose of the placement of the acupuncture to stimulate the sensory division of the trigeminal nerve, as well as sensory branches of cervical nerves.

Certain embodiments described herein involve a self administrative device and method to accomplish and improve on the effects and goals of acupuncture. Self administration of the acupuncture affords a timely application during the prodromal period so the full evolution of the headache can be aborted. Patients who suffer from migraines often feel a prodrome but do not have the time to see a physician who is skilled in acupuncture, or an acupuncture therapist. This is also an expensive endeavor for the patient, as acupuncture generally is costly. When delays in acupuncture are applied the therapy has been thought to be, by the inventor, much less effective. Although the acupuncture can be applied during the pain period to relieve pain, the most effective and the best performance of acupuncture is as an abortive function to prevent the development of the full headache disorder. Another improvement consists of accomplishing a critical level of irritation and stimulation in order to elicit a triple inflammatory response which will augment the effects of acupuncture.

In the case of migraine, the application of acupuncture is thought to suppress spontaneous neuronal activity theorized as causing the severe pain and often stimulating the nausea and vomiting typical of the migraine syndrome.

### Current Therapeutic Problems

The current therapy of tension migraine headache has been usually treated pharmacologically. Dissociated side effects of these agents can be problematic for patients. These agents are often extensively expensive, require prescriptions, often require many trial and error courses of medications, and need to be renewed. The use of a self administrating device which has no systemic side effects is a clear improvement in the treatment of human migraine and related headache disorders. The use of a self administrating device has a low cost, has no substantial systemic side effects, and can be applied in repeated locations. Among professional organizations there has been endorsement of acupuncture therapy as being effective. The self administration device allows an improvement over existing administration methods which require another professional person, physician or acupuncturist to place the needles. The acupuncturist also is using the treatment to achieve prophylactic effects as the application is not synchronized with the onset of the headache during the prodrome. This approach severely reduces the impact of this modality on the course of headache morbidity. The use of a self administered acupuncture with the devices described herein solve this problem and allow a more effective application of the treatment modality, while simultaneously allowing a more convenient therapy for the afflicted. The timely application of the needle penetration synchronized at the headache prodrome allows for potentiating and improvement of needle skin puncture effectiveness in the treatment of human headache disorders. In certain embodiments, the therapy is administered during the prodromal phase of the headache so that the painful portion of the headache does not fully develop and the person afflicted with the migraine does not experience most of the pain related morbidity.

### The Temporal Sequence of Migraine Headache

Common migraine is conventionally episodic in nature. The discrete severely painful episodes however can be anticipated by the suffer during the prodrome, a period in which an afflicted individual feels malaise, early nausea, loss of appetite, increasing response to sensory stimuli such as odors, sound, light, and increasing muscle tension. The prodrome usually is not associated with significant pain which is the most debilitating of the migraine syndrome. In a minority of patients, the afflicted may experience a visual aura consisting of flashing lights, visual hallucination usually consisting of zig-zag lines, shimmering vision or transient blind spots. Variations occur in the prodrome experience and in visual auras. About 15% of migraine sufferers experience a visual aura however a much larger number (>80%) experience a prodrome. The prodrome period allows the use of abortive medications and activities. Some patients use cold packs, pressing bands, warms packs, message and postures to try to relieve the severity of the impending pain trying to avoid use abortive pharmaceuticals.

Methods and devices are disclosed herein, which automatically treat headaches during the prodrome period of migraine, with the purpose of aborting a migraine headache or reducing its severity. The methods and devices, may be used during the prodrome of migraine, or may be used for other headaches listed in Table 1 in which a prodrome is experienced.

### Overlap Between Migraine and Tension Headache

Persons of skill in the art realize that there may be an overlap in the diagnosis between migraine and tension headache. Some patients with episodic migraine will have frequent tension headaches, and some with frequent tension headaches will have migraine. Although migraine is typically associated with nausea—some migraines are not—and are instead predominately characterized by a severe intensity, a throbbing quality, and intense photophobia associated with autonomic symptoms of pallor of skin, sweating, flushing. In these situations, the headache may still be classified as a migraine. Tension headaches are generally less severe and characterized by band like tightness around the head, neck stiffness, jaw stiffness, often relieved by mild analgesics such as aspirin, indocin or other non-steroidal anti-inflammatory drugs.

Peri-Orbital Tactile Suppression of Photophobia with Benign Essential Blepharospasm and Further Insight into Sensory Tricks

Photophobia has been recognized as an important component of benign essential blepharospasm. (See e.g., Borodic G E Quigg C, Photophobia and Essential Blepharospasm, American Society of Ophthalmic Plastic and Reconstructive Surgeons, Annual Meeting, Chicago 1996; Adams A H, Digre K B, Bhupendra C, Patel K, Anderson R L, Warner J E, Katz B J, The evaluation of light sensitivity in benign essential blepharospasm, Am J Ophthalmol 142:82-87, 2006; Herz N L, Yen M T, Modulation of sensory photophobia in essential blepharospasm with chromatic lenses, Ophthalmology 112:2208-2211, 2005; Borodic G E, Acquadro M, Johnson E., Botulinum toxin for the treatment of pain and inflammation, Expert Opin Investig Drugs 10(8): 1531-1544, 2001, the disclosures of which are incorporated by reference herein in their entireties). The incidence of this symptom is present in over 80% of patients, and is often noted to be particularly severe during exacerbations of this chronic disease. Relief has been traditionally achieved by patents with a filtering lens, and more recently, with use of specialized filtering lenses which are more absorbing in the shorter wavelength spectrum. Additionally, patients have related increased light sensitivity with the need for repeat botulinum toxin injections after 10-12 weeks, and many note improvement in photophobia with subsequent botulinum toxin administration. The use of botulinum toxin for the treatment of migraine and other forms of essential headaches, both often associated with intermittent and sometimes chronic photophobia, has been extensively studied with successful results in many but not all studies. From these observations, it is reasonable to induce possible relationships between photophobia and the basic neural mechanisms underlying eyelid involuntary movement disease and possible treatment effects.

The “sensory trick” is a common behavior in the bilateral blepharospasm and Meige syndrome patient. Typically, a digital effort is made unilaterally with elevation of the brow causing the eyelid to stay open long enough to improve visual function. Both the patient and the examiner initially interpret the behavior as simply prying open an eyelid afflicted with involuntary spasmodic movement to obtain vision. This explanation, however, is incomplete because the maneuver benefits contralateral eyelid movement and creates a sensory change. Elevation of one eyebrow results in improvement in blepharospasm in the contralateral eye may provide some insight into disease mechanism. Usually, the sensory trick is location specific and unilateral for each patient. Improvement in the contralateral eyelid is clearly not mechanically based, but rather, is dependent on the suppression brainstem function or other central nervous system structures governing the blink reflex. In other medical centers, stimulating devices have recently been tried to improve control of eye movements mimicking the self-administered tactile effort.

Using studies involving the trigeminal-facial motor reflex, Tolosa et al. described increased length and amplitudes in the R2 wave measured in the orbicularis contralateral to the stimulated side stimulated side in patients with essential blepharospasm, and interpreted these findings as representing a population of unstable brainstem interneurons governing spontaneous blinking. These authors have subsequently demonstrated the effect of tactile stimulation of the brow causes a suppression of these R2 wave abnormalities. Others have demonstrated suppression of the R2 waves using repetitive periorbital electrical sensory stimulation. However, before this disclosure, no author has recognized or described the tactile effect on subjective visual perception of brightness. See Hallet M, Evinger C, Janovic J, Stacy M. Update on blepharospasm: report from the BEBRF International Workshop. Neurology. 2008; 71:1275-1282; Tolosa E, Monsterrat L, Bayes A. Blink reflex studies in focal dystonias: enhanced excitability of brainstem interneurons in cranial dystonia and spasmodic Torticollis. Mov Disord. 1988; 3:61-69; Gómez-Wong E, Marti M J, Cossu G. Fabregat N, Tolosa E, VallsSolé J. The ‘geste antagonist’ induces transient modulation of the blink reflex in human patients with blepharospasm. Neurosci Lett. 1998; 251:125-128; Gómez-Wong E, Marti M J, Tolosa E, Valls-Solé J. Sensory modulation of the blink reflex in patients with blepharospasm. Arch Neurol. 1998; 55:1233-1237; and Sommer M, Ferbert A. The stimulus intensity modifies the blink reflex recovery cycle in healthy subjects and in blepharospasm. Clin Neurophysiol. 2001; 112:2293-2299, the disclosures of which are herein incorporated by reference.

From observations and patient interviews, it was observed that the sensory trick suppresses photophobia. Patients having a strong predisposition to brow and periorbital self stimulation relate an almost instantaneous decreased in light sensitivity which usually correlates to reduction in intensity of symptoms. The reduction can be demonstrated when light intensity is increased in either eye, that is, in the eye receiving the tactile stimulation and in the contralateral eye. The reduction in photophobia ceases simultaneous on withdrawal of tactile stimulation. This phenomenon was observed in a series of 18 cases of essential blepharospasm and Meige disease. That observation contributed to the theory and motivation for implantation of self drilling titanium fixation screws (fixation pins) for both partial orbicularis restrictive immobilization and sensory augmentation over the trigeminal nerve during routine facial movements. The sensory augmentation may suppress blink interneurons simulating a sensory trick. Patients with implantable metallic pins around the orbital rim causing orbicularis fixation improve with respect to both blepharospasm and photophobia over a sustained period. The pins cause a slight increased sensitivity to the brow region as well as alter movement pattern of facial muscles with attendant alteration in proprioception in the region. The success achieved with this procedure lends credibility to the notion that certain facial movement disorders may be altered by manipulating certain afferent sensory inputs and that there is significant interplay between visual and somatic sensory systems causing this condition.

Without being bound by theory, it is believed that the mechanism for tactile suppression of photophobia potentially relates to suppression of centers in the brainstem receiving extra geniculate retinal projections governing the sensation of brightness and involuntary and automatic eyelid closure. Such neurons may be associated with pretectal region known for regulating the pupillary light reflex and along retinal projections into brainstem regulating automatic eye tracking and lid movements.

Although migraine is a different syndrome than essential blepharospasm, similarities in the benefit of tactile stimulation is apparent for both conditions, and the morbidity of both conditions can be viewed as a result of sensitized central neurons. In the case of blepharospasm, hyper excitable sensitized neurons control blink movements, whereas in the case of migraine, sensitized neurons govern pain perception and are located in the thalamus and associated regions. A needle stimulator causing the triple response of Lewis has been noted to improve both migraine pain and abnormal blink movements associated with essential involuntary neurologic blepharospasm.

Both conditions (migraine and blepharospasm), can be treated with various embodiments of the methods and devices described herein. Moreover, photophobic migraine and other chronic headache sufferers are expected to be an important selection criterion for use of embodiments of the methods and devices described herein.

### Light and the Generation of Migraine and Related Headache Pain

Photophobia is a well appreciated component of the migraine and tension headache syndrome. During periods of severe pain, light is perceived as uncomfortable and very annoying. Patients often seek a dark room to enhance comfort.

More specifically, light intensity has been perceived to make the headache worse. Without being bound by theory, this observation indicates that the perception of light plays a role in the generation of migraine headache pain, and that light sensitivity may not just be an epiphenomena. Retinal projections into the brain pass through the geniculate body are generally appreciated as image related signals which project to occipital cortex and associated brain areas that process and recognize images. Retina ganglion cells also project through extra-geniculate regions of the brain which effects changes in papillary size and papillary light reflexes, blink, and circadian rhythms. In these regions, thalamic pain centers can integrate information on light intensity and light signal intensity can aggravate excitability of thalamic pain neurons associated with migraine. Hypothalamic functions are closely associated with extra geniculate retinal signaling as well as regulation of hormonal menstrual cycles via release of small peptide release factors into the pituitary gland. Migraine is much more common during early menstruation than any other time of the cycle, indicating a neuro-endocrine link to the pathogensis. As disclosed herein for the first time, it was unexpectedly observed that stimulation of the scalp brow and forehead region can consistently depress photophobia associated with a facial movement disease know as benign essential blepharospasm and Meige syndrome. Self administered pinprick eliciting a triple response of Lewis was especially effective in depresses and temporarily eliminating photophobia in this condition. These movement diseases are also associated with hyperexcitable brainstem interneurons.

Without being bound by theory, and by using a logical mathematical substitution of phenomena, it is disclosed herein for the first time, that self administered pinprick can create depression of hyperexcitable interneurons caused or aggravated by light stimulation as projected through extra-geniculate pathways from the retina. The generation of the migraine syndrome is no doubt multifactorial, however, dampening the sensitization by depressing light sensitivity appears to be important in mitigating pain.

Prior to this disclosure, the association between somatic stimulation and light sensitivity from the human eye in certain pathologic conditions has not been made. The inverse relationship is pivotal to the practice of the invention as stimulation using tactile devices, increased stimulation using a penetrating needle or microblade (which in one embodiment can be an implantable device, for example a metallic screw) eliciting a triple response of Lewis which functions to enhance the suppression of light sensitivity. This in kind decreases sensitization of central pain neurons. Treatment of photophobia by somatic sensory stimulation in order to abort migraine and related headaches is accomplished using embodiments of devices and methods described herein.

### Use of an Implantable Self-Stimulation Implant Attached to Outer Bone Table for Automatic Stimulation of and Desensitization of the Trigeminal Nerve Using Natural Facial Movement During Facial Expression

In one embodiment, a method of stimulating the trigeminal nerve branches to achieve desensitization involves the use of an implant which tethers dermal and attached facial muscular elements to underlying bone. In one embodiment, the implant can be placed with a micro incision in the region of the scalp, forehead, brow, occipital area, face, posterior auricular region with the intention of attaching muscular and dermal elements to bone. During natural facial movements, alteration in the sensory nerves produced by this type of fixation can serve to desensitize the trigeminal nerve to provide for a spontaneous, constant effect not requiring self stimulation or any conscious effort by the patient treated for migraine or related headache disorders. With natural facial movement and facial expression, this type of tissue tethering has been shown to decrease photophobia, an important component of the migraine syndrome. The implant is fixated to bone and can be applied in regions not containing critical vessels and motor nerves to avoid complications of hemorrhage or paralysis. The effect will create an internal type of neurogenic inflammation mimicking the triple response of Lewis, involving stimulating and desensitizing the trigeminal nerve, so that this nerve would be less prone to contribute to sensitization in conjunction with central nervous system sensitization within the thalamus and related brain structures so that evolution of migraine pain is mitigated or aborted.

In one embodiment of a component of a method of migraine treatment, a 340 mm, preferably a 3-7 mm screw, and most preferably a 4-5 mm screw is placed in certain areas of the forehead, brow, face and scalp to provide dermal muscle fixation and stimulation of trigeminal nerve sensory elements using a tightly fixating wrench through 1-2 mm incisions impaling dermal elements, muscular elements and placing a tight fixation by compressing these elements firmly against underlying bone. In certain embodiments, the screw is titanium. Minimal closure effort is made with one or two sutures. During the healing process, the fixation creates an altered sensation and a sensory stimulus simulating self administered pressure or stimulation often applied by the patient who suffers from migraine or related headaches.

One implementation is most useful in treating those afflicted with chronic recurrent headaches making intermittent self stimulation on the skin surface difficult and likely effective because of frequent applications. Migraine patients with frequent attacks often undergo central sensitization which can not easily be treated by self stimulation during the prodromal periods, so that a more robust method of stimulation requiring this fixation implant who be a preferred method of therapy. This application would be best applied in migraine with severe chronic daily headaches or severe forms of chronic myofascial headaches.

In certain embodiments, the screw and/or pin placement can be anywhere along the facial bone, but most preferably in the periorbital area. The pin can be placed at angles or variable depths to achieve maximal effect or sensory nerve stimulation. Pin placement in close proximity to the supra-orbital nerve gives maximal stimulating effect. Pins once surgically placed may be stimulated by external devices (for example, electrical or ultrasonic devices or their equivalents as is known in the art) to enhance effects on sensory nerve stimulation.

In certain embodiments, a method for treating chronic recurrent migraine or related headaches in a subject comprises surgically implanting a device through a micro incision in the subject's skin. The device causes a fixation of the subject's dermis and muscle elements to the subject's bone, which desensitizes the trigeminal nerve. In certain embodiments, a method for treating chronic severe tension or related headaches in a subject comprises surgically implanting a device through a micro incision in the subject's skin. In certain embodiments, a method for treating recurrent cluster headaches in a subject comprises surgically implanting a device through a micro incision in the subject's skin. In certain embodiments, a method for treating recurrent migraine and associated headaches in a subject comprises surgically implanting a device through a micro incision in the subject's skin.

In certain preferred embodiments, the device comprises a metallic screw, and the method further involves compressing the subject's soft tissues to the subject's bone with engagement of the screw to the bone. In certain preferred embodiments, the metallic screw is titanium and measures between 4-5 mm. In certain preferred embodiments, the metallic screw is stainless steel. In certain preferred embodiments, the metallic screw is magnetic.

In certain preferred embodiments, the device is implanted in multiple locations.

In certain preferred embodiments, the related headaches are severe myofascial pain, cluster headaches, cervciogenic headaches, headaches associated with fibromyalgia, or tension headaches.

In certain preferred embodiments, the method involves application of ultrasonic or electronic energy to enhance stimulation of the trigeminal nerve.

In certain embodiments, the invention is directed to a method for treating photophobia in a subject by implanting a metallic device fixated to bone in proximity to the subject's facial sensory nerves.

### Example 14

A 54 year old woman underwent an implantation procedure of a peri-orbital and brow pin for neurologic blepharospasm. She also suffered from chronic tension and occasional migraine headaches. She noted a sensory trick which suppressed her involuntary movements. She underwent a brow pinning procedure which fixated her forehead scalp to the underlying osseous bone. Her involuntary blepharsospasm improved over subsequent months. Although she suffered a usual mild post procedure headache, she unexpectedly and surprisingly benefited from the decreased incidence of subsequent tension and migraine headaches. She further noted that her general light sensitivity was improved and her ability to sustain her employment without absence from headache pain was increased.

### Use of an Implantable Self-Stimulation Implant not Attached to Outer Bone Table for Automatic Stimulation of and Desensitization of the Trigeminal Nerve Using Natural Facial Movement During Facial Expression

In other embodiments, an object is to provide sensory nerve stimulation from an implant placed in close proximity to sensory nerves which stimulates the nerves during natural human movement and therefore, suppresses central pain centers in the brainstem, spine, or related brain areas for the purpose of mitigating symptoms of chronic pain. The surgical implant in certain circumstances may not have to attach to the subject's bone, but rather be placed in close proximity to a sensory nerve which during the course of natural facial movement, creates a point of stimulation of the sensory nerve, which causes central desensitization of pain centers in the brainstem or other associated pain centers in the central nervous system. Such an application of the implant exploits natural facial or other human movement occurring during blinking, facial expression, chewing, breathing, or the like to stimulate the sensory nerves in order to create an enhanced sensory impulse into the central nervous system which has the effect of suppressing pain associated with headache syndromes.

These methods and devices are not limited to head and neck, but work on other body regions which are afflicted with pain associated with tactile suppression, such as rubbing pressing, messaging, or touching, for example, myofascial pain, fibromyalgia (diffuse or generalized), arthritis and arthralgias, and muscle spasm related pain.

In various embodiments, the implant can be metallic, for example titanium, gold, stainless steel or any other tissue inert alloy. Ceramic implants are possible so long as the materials can create a sensory stimulation during movement. The implant may be an absorbable substance to as to not require potential future removal. Such substances may include but not limited to nylon, catgut, absorbable cloth fiber, silk, poly-L-lactic acid, polyglycolic acid, polymethyl methacrylate, and the like.

In some embodiments, absorbable implants may cause a limited inflammatory response which can further enhance sensory nerve stimulation during natural facial movement. Any implant used for this purpose can be placed at any tissue plane in close proximity to sensory nerves. Sensory nerves on the face include the trigeminal nerve (all divisions), cervical nerves, sensory division of facial nerve, and sensory nerves of the mandibular bone. This approach is not limited to the face, head and neck.

In other embodiments, the implant is partially exteriorized so as to resemble a body piercing with a pin, ring, sphere, or any ornate metallic figure for aesthetic camouflage. Exteriorized implants also may further provide enhanced sensory stimulation because of proximity to superficial cutaneous nerve endings. An example of a none bone fixed implant for the treatment of chronic tension and chronic migraine is given in the example below.

### Example 15

A 53 year old woman experienced blepharospasm and severe chronic migraine and chronic tension headaches (myofascial pain headaches and neck aches). She did not wish a bone fixed implant. A gold metallic brow ring, usually used for aesthetic appeal, was recommended and therefore placed through the brow in closure proximity to the supra-orbital nerve (first division of trigeminal nerve). She experienced marked improvement in the severity and frequency of her headache syndrome and continued to wear the exteriorized soft tissue ring for several years.

Table 1: Headaches with which the Implantable Device May be Used (1988 Headache and Facial Pain Classification System by the International Headache Society; Classification of Headache Disorders, Cranial Neuralgias, and Facial Pain)
1. Migraine
   1.1 Migraine without aura
   1.2 Migraine with aura
      1.2.1 Migraine with typical aura
      1.2.2 Migraine with prolonged aura
      1.2.3 Familial hemiplegic migraine
      1.2.4 Basilar migraine
      1.2.5 Migraine aura without headache
      1.2.6 Migraine with acute onset aura
   1.3 Ophthalmoplegic migraine
   1.4 Retinal migraine
   1.5 Childhood periodic syndromes that may be precursors to or associated with migraine
      1.5.1 Benign paroxysmal vertigo of childhood
      1.5.2 Alternating hemiplegia of childhood
   1.6 Complications of migraine
      1.6.1 Status migrainosus
      1.6.2 Migrainous infarction
   1.7 Migrainous disorder not fulfilling above criteria
2. Tension type headache
   2.1 Episodic tension-type headache
      2.1.1 Episodic tension-type headache associated with disorder of pericranial muscles
      2.1.2 Episodic tension-type headache unassociated with disorder of pericranial muscles
   2.2 Chronic tension-type headache
      2.2.1 Chronic tension-type headache associated disorder of pericranial muscles
      2.2.2 Chronic tension-type headache unassociated with disorder of pericranial muscles
   2.3 Headache of the tension-type not fulfilling above criteria
3. Cluster headache and chronic paroxysmal hemicrcrania
   3.1 Cluster headache
      3.1.1 Cluster headache periodicity undetermined
      3.1.2 Episodic cluster headache
      3.1.3 Chronic cluster headache
         3.1.3.1 Unremitting from onset
         3.1.3.2 Evolved from episodic
   3.2 Chronic paroxysmal hemicrania
   3.3 Cluster headache-like disorder not fulfilling above criteria
4. Miscellaneous headaches unassociated with structural lesion
   4.1 Idiopathic stabbing headache
   4.2 External compression headache
   4.3 Cold stimulus headache
      4.3.1 External application of a cold stimulus
      4.3.2 Ingestion of a cold stimulus
   4.4 Benign cough headache
   4.5 Benign exertional headache
   4.6 Headaches associated with sexual activity
      4.6.1 Dull type
      4.6.2 Explosive type
      4.6.3 Postural type
5. Headache associated with head trauma
   5.1 Acute posttraumatic headache
      5.1.1 With significant head trauma and/or confirmatory signs
      5.1.2 With minor head trauma and no confirmatory signs
   5.2 Chronic posttraumatic headache
      5.2.1 With significant head trauma and/or confirmatory signs
      5.2.2 With minor head trauma and no confirmatory signs
6. Headache associated with vascular disorders
   6.1 Acute ischemic cerebrovascular disease
      6.1.1 Transient ischemic attack (TIA)
      6.1.2 Thromboembolic stroke
   6.2 Intracranial hematoma
      6.2.1 Intracerebral hematoma
      6.2.2 Subdural hematoma
      6.2.3 Epidural hematoma
   6.3 Subarachnoid hemorrhage
   6.4 Unruptured vascular malformation
      6.4.1 Arteriovenous malformation
      6.4.2 Saccular aneurysm
   6.5 Arteritis
      6.5.1 Giant cell arteritis
      6.5.2 Other systemic arteritides
      6.5.3 Primary intracranial arteritis
   6.6 Carotid or vertebral artery pain
      6.6.1 Carotid or vertebral dissection
      6.6.2 Carotidynia (idiopathic)
      6.6.3 Post endarterectomy headache
   6.7 Venous thrombosis
   6.8 Arterial hypertension
      6.8.1 Acute pressor response to exogenous agents
      6.8.2 Pheochromocytoma
      6.8.3 Malignant (accelerated) hypertension
      6.8.4 Preeclampsia and eclampsia
   6.9 Headache associated with other vascular disorder
7. Headache associated with nonvascular intracranial disorder
   7.1 High cerebrospinal fluid pressure
      7.1.1 Benign intracranial hypertension
      7.1.2 High-pressure hydrocephalus
   7.2 Low cerebrospinal fluid pressure
      7.2.1 Post lumbar puncture headache
      7.2.2 Cerebrospinal fluid fistula headache
   7.3 Intracranial infection
   7.4 Intracranial sarcoidosis and other noninfectious inflammatory diseases
   7.5 Headache related to intrathecal injections
      7.5.1 Direct effect
      7.5.2 Due to chemical meningitis
   7.6 Intracranial neoplasm
   7.7 Headache associated with other
8. Headache associated with substances or their withdrawal
   8.1 Headache induced by acute substance use or exposure
      8.1.1 Nitrate/nitrite induced headache
      8.1.2 Monosodium glutamate induce
      8.1.3 Carbon monoxide induced headache
      8.1.4 Alcohol-induced headache
      8.1.5 Other substances
   8.2 Headache induced by chronic substance use or exposure
      8.2.1 Ergotamine induced headache
      8.2.2 Analgesics abuse headache
      8.2.3 Other substances
   8.3 Headache from substance withdrawal (acute use)
      8.3.1 Alcohol withdrawal headache (hangover)
      8.3.2 Other substances
   8.4 Headache from substance withdrawal (chronic use)
      8.4.1 Ergotamine withdrawal headache
      8.4.2 Caffeine withdrawal headache
      8.4.3 Narcotics abstinence headache
      8.4.4 Other substances
   8.5 Headache associated with substances but with uncertain mechanism
      8.5.1 Birth control pills or estrogens
      8.5.2 Other substances
9. Headache associated with noncephalic infection
   9.1 Viral infection
      9.1.1 Focal noncephalic
      9.1.2 Systemic
   9.2 Bacterial infection
      9.2.1 Focal noncephalic
      9.2.2 Systemic (septicemia)
   9.3 Headache related to other infection
10. Headache associated with metabolic disorder
   10.1 Hypoxia
      10.1.1 High-altitude headache
      10.1.2 Hypoxic headache (law-pressure environment, pulmonary disease causing hypoxia)
      10.1.3 Sleep apnea headache
   10.2 Hypercapnia
   10.3 Mixed hypoxia and hypercapnia
   10.4 Hypoglycemia
   10.5 Dialysis
   10.6 Headache related to other metabolic abnormality
11. Headache or facial pain associated with disorder of cranium, neck, eyes, ears, nose, sinuses, teeth, mouth or other facial or cranial structures
   11.1 Cranial bone
   11.2 Neck
      11.2.1 Cervical spine
      11.2.2 Retropharyngeal tendinitis
   11.3 Eyes
      11.3.1 Acute glaucoma
      11.3.2 Refractive errors
      11.3.3 Heterophoria or heterotropia
   11.4 Ears
   11.5 Nose and Sinuses
      11.5.1 Acute sinus headache
      11.5.2 Other diseases of nose or sinuses
   11.6 Teeth, jaws and related structures
   11.7 Temporomandibular joint disease (functional disorders are coded to group 2)
12. Cranial neuralgias, nerve trunk pain, and deafferentation pain
   12.1 Persistent (in contrast to tic-like) pain of cranial nerve origin
      12.1.1 Compression or distortion of cranial nerves and second or third cervical roots
      12.1.2 Demyelination of cranial nerves
         12.1.2.1 Optic neuritis (retrobulbar neuritis)
      12.1.3 Infarction of cranial nerves
         121.3.1 Diabetic neuritis
      12.1.4 Inflammation of cranial nerves
         12.1.41. Herpes Zoster
         12.1.4.2 Chronic postherpetic neuralgia
      12.1.5 Tolosa-Hunt syndrome
      12.1.6 Neck-tongue syndrome
      12.1.7 Other causes of persistent pain of cranial nerve origin
   12.2 Trigeminal neuralgia
      12.2.1 Idiopathic trigeminal neuralgia
      12.2.2 Symptomatic trgeminal neuralgia
         12.2.2.1 Compression of trigeminal root or ganglion
         12.2.2.2 Central lesions
   12.3 Glossopharyngeal neuralgia
      12.3.1 Idiopathic glossopharyngeal neuralgia
      12.3.2 Symptomatic glossopharyngeal neuralgia
   12.4 Nervus intermedius neuralgia
   12.5 Superior laryngeal neuralgia
   12.6 Occipital neuralgia
   12.7 Central causes of head and facial pain other than tic douloureux
      12.7.1 Anaesthesia dolorosa
      12.7.2 Thalamic pain
   12.8 Facial pain not fulfilling criteria in groups 11 and 12
13. Headache not classifiable

The patent applications and other literature referred to in the instant specification are incorporated herein by reference in their entireties. The description of the embodiments, examples, and figures herein are given by way of example, and not by way of limitation. A person having ordinary skill in the relevant art is a medical doctor.

I claim:

1. A surgical method for elevating and/or contouring facial soft tissue in a human patient in need thereof comprising altering or reinforcing attachment of muscle to underlying skull or facial bone by one or both of (a) placing a screw through microincisions or (b) injecting a bioadhesive to the undersurface of said muscle, thereby fixating said muscle and overlying facial soft tissue to said underlying skull or facial bone;
wherein said facial soft tissue is elevated and/or a contour, a proportion, dynamic facial wrinkles, static facial wrinkles, and a movement pattern of said patient's face are redefined.

2. The surgical method of claim 1, wherein altering or reinforcing attachment of muscle to underlying skull or facial bone comprises (a) placing a screw through microincisions but not (b) injecting a bioadhesive to the undersurface of said muscle.

3. The surgical method of claim 1, wherein altering or reinforcing attachment of muscle to underlying skull or facial bone comprises (b) injecting a bioadhesive to the undersurface of said muscle but not (a) placing a screw through microincisions.

4. The surgical method of claim 1, wherein said muscles are selected from the group consisting of frontalis, zygomaticus major, zygomaticus minor, platysma, orbicularis oculi, orbicularis ori, buccinators, mentalis, glaellar and any combination of other muscles overlaying facial or skull bones.

5. The surgical method of claim 1, comprising multiple points of fixation.

6. The surgical method of claim 1, wherein said elevated and/or contoured facial soft tissue comprises the brow or forehead of said patient.

7. The surgical method of claim 1, wherein said elevated and/or contoured facial soft tissue comprises the midface or cheek region of said patient.

8. The surgical method of claim 1, wherein said elevated and/or contoured facial soft tissue comprises the jowl region of said patient.

9. The surgical method of claim 1, wherein said elevated and/or contoured facial soft tissue comprises the submental region or neck region of said patient.

10. The surgical method of claim 1, wherein redefinition of said facial soft tissue comprises elevation of the cheeks, forehead and shrinkage of lower facial structures; thereby enhancing an upper face volume and diminishing a lower face volume.

11. The surgical method of claim 1, wherein said bioadhesive is a cyanoacrylate.

12. The surgical method of claim 11, wherein said cynaoacrylate induces and inflammatory response in less than about 0.5% of patients.

13. The surgical method of claim 1, wherein injection of bioadhesive is made through a needle or highly tapered injection device which can be administered with a small puncture wound.

14. The surgical method of claim 13, wherein a tip of the injection is in the fibrofatty plane between facial muscle and bone, thereby creating a new muscle to bone adhesion not previously present.

15. A surgical method for elevating and/or contouring soft tissue in a brow region in a human patient afflicted with blepharospasm comprising one or both of (a) placing a screw through microincisions in the subcutaneous connective tissue, fascia, and muscle into an underlying facial, orbital or frontal bones or (b) injecting a bioadhesive to an undersurface of said tissue, thereby fixating said tissue to said underlying facial, orbital or frontal bones; wherein said soft tissue in a brow region is elevated and/or contoured.

16. The surgical method of claim 15, wherein elevating and/or contouring tissue within a brow region comprises (a) placing a screw through microincisions but not (b) injecting a bioadhesive.

17. The surgical method of claim 15, wherein elevating and/or contouring tissue within a brow region comprises (b) injecting a bioadhesive but not (a) placing a screw through microincisions.

18. A device comprising a metallic or ceramic screw shaft and a compression head, which when applied over the skin or through an incisional site, engages said screw shaft into facial bone to fixate facial muscle and decrease contractility of said muscle and induce sub-muscular scarring and adhesion of said muscle to facial bone to reduce or decrease static and dynamic facial lines or wrinkles.

19. A surgical method for elevating and/or contouring facial soft tissue in a human patient in need thereof comprising altering or reinforcing attachment of muscle to underlying skull or facial bone comprising injecting a bioadhesive to the undersurface of said muscle, thereby fixating said muscle and overlying facial soft tissue to said underlying skull or facial bone;
wherein said facial soft tissue is elevated and/or a contour, a proportion, and a movement pattern of said patient's face are redefined.
